(19) United States
(12) Patent Application Publication
(10) Pub. No.: US 2004/0063725 A1
(43) Pub. Date: Apr. 1, 2004

(54) Novel n(phenylsulphonyl)glycine derivatives and their therapeutic use

(76) Inventors: Martine Barth, Asnieres Les Dijon (FR); Michel Bondoux, Fontaine Les Dijon (FR); Christophe Matt, Neuville Sur Saone (FR); Pierre Dodey, Fontaine Les Dijon (FR); Jean-Michel Luccarini, Dijon (FR); Jean-Luc Paquet, Brognon (FR); Didier Pruneau, Pasques (FR)

Correspondence Address: MERCHANT & GOULD PC, P.O. BOX 2903, MINNEAPOLIS, MN, 55402-0903, US

(21) Appl. No.: 10/451,617

(22) PCT Filed: Jan. 7, 2002
(86) PCT No.: PCT/FR02/00033

(30) Foreign Application Priority Data
Jan. 8, 2001 (FR) 01/00195

(51) Int. Cl. A61K 31/496; A61K 31/455; A61K 31/445; A61K 31/4172; A61K 31/18

(52) U.S. Cl. 514/255.01; 514/317; 514/356; 514/400; 514/602; 544/386; 546/225; 546/316; 546/315; 548/338.1; 564/84

(57) ABSTRACT

The present invention relates to novel N-(phenylsulphonyl)glycyl-glycine compounds, which are defined by formula I and the description, as well as their method of preparation and their use in therapeutics

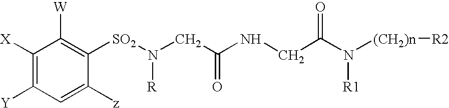

The present invention relates to novel N-(phenylsulphonyl)glycine compounds, their method of preparation and their use for obtaining pharmaceutical compositions.

These novel compounds are useful in therapeutics, particularly for treating pain.

### PRIOR ART

Compounds having an arylsulphonamide-type group and glycine in their structure are already known. N-α-arylsulphonylaminoacyl-p-amidinophenyl-alaninamides, which are selective inhibitors of thrombin and which are useful as antithrombotics, can be cited for example, from EP 236 163 and EP 236 164. Compounds of structure quite close to the preceding ones, simultaneously comprising an arylsulphamoyl group and a substituted phenylamidine group, which have the property of binding to neuropeptide Y receptors and which can be useful for treating hypertension, angina, atherosclerosis, depression, anxiety, inflammation, allergy or fatty excess weight, are also known, from EP 614 911.

EP 558 961 also suggests the use of substituted arylsulphonamide amino acid type compounds for treating thrombosis due to their anticoagulant properties.

Studies in relation to the antithrombotic properties of compounds possessing an arylsulphonamide group and a phenylamidine group in their structure have also been published in Pharmazie, 1984, vol. 39 (5), pages 315-317 and in Pharmazie, 1986, vol 41 (4), p. 233-235.

In the same field of pharmacological activity, WO 92/16549 A1 describes phenylalanine derivatives having an arylsulphonamide group, which are inhibitors of proteinase, notably inhibitors of thrombin.

Compounds of N-(arylsulphonyl)amino acid structure, which are useful for treating inflammatory illnesses, are also known, from WO 97/25315.

In a different therapeutic field, WO 00/34313 describes peptides which can comprise a chain-end arylsulphonyl group and which are claimed for their ability to inhibit procollagen-C-proteinase. Compounds of close structure, which are presented as inhibitors of porcine pancreatic elastase, are also known from the publication J. Chem. Soc., Perkin Trans. 1(1986), (9), p. 1655-64.

AIMS OF THE INVENTION

The invention relates to novel compounds comprising a substituted N-(arylsulphonyl)glycyl-glycine chain, said compounds being useful notably as active principles of medicaments which are intended for treating pain, and particularly hyperalgiae and major algesiae.

### DESCRIPTION

An N-(phenylsulphonyl)glycine compound is proposed, according to the present invention, as a novel industrial product, said compound being characterised in that it is selected from the whole which is made up of:

i) compounds of formula:

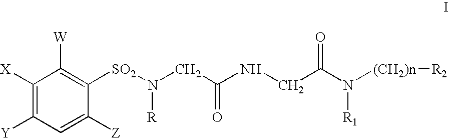

in which

W represents a chlorine atom,

X represents a hydrogen atom, a methyl group or a chlorine atom,

Y and Z independently each represent a hydrogen atom or a chlorine atom, or

X and W or X and Y, together with the carbon atoms to which they are bound, form a phenyl ring,

R represents a hydrogen atom, an allyl group or a C₁-C₄ alkyl group which is non-substituted or substituted with a phenyl group, a methoxy group, a pyridinyl group, a carboxamide group or an N-methylcarboxamide group,

R₁ represents a hydrogen atom, a C₁-C₄ alkyl group or

a (CH₂)ₘ—R′₂ group,

n and m independently each represent 1, 2, 3 or 4,

R₂ and R′₂ independently each represent

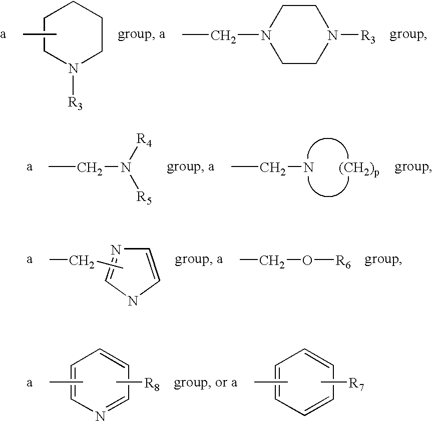

group, and

R₃ represents a hydrogen atom or a C₁-C₄ alkyl group,

R₄ represents a hydrogen atom, a COCH₃ group, a COOCH₃ group, or a C₁-C₄ alkyl group,

R₅ represents a hydrogen atom or a C₁-C₄ alkyl group, which is non-substituted or substituted with a phenyl group,

R₆ represents a hydrogen atom or a CONHC₂H₅ group,

R₇ represents a hydrogen atom,

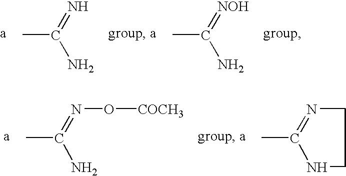

group or a CONHCH₃ group,

R₈ represents a hydrogen atom, an NH₂ group, or a C₁-C₄ alkyl group, and

p=4, 5 or 6,

ii) addition salts of the compounds of formula I above with an acid.

A method of preparation of the compounds of formula I, as well as of their addition salts, is also recommended, according to the invention.

The use is also recommended of a substance which is selected from the compounds of formula I and their non-toxic addition salts for preparing a medicament, which is useful in human or animal therapeutics, and which is intended for preventing or for treating pathologies linked to pain, notably hyperalgiae following an inflammatory state or major algesiae linked to other pathological states, such as cancer, for example.

### DETAILED DESCRIPTI N

In formula I, “C₁-C₄ alkyl group” is to be understood as a hydrocarbon chain having 1 to 4 carbon atoms, which is linear or branched, or even cyclic.

A C₁-C₄ alkyl group is for example a methyl, ethyl, propyl, butyl, 1-methylethyl, 1-methylpropyl, 2-methylpropyl, 1,1-dimethylethyl or a cyclopropylmethyl group.

“A C₁-C₄ alkyl group substituted with a phenyl group” is to be understood as meaning a C₁-C₄ alkyl group one of the hydrogen atoms of which is substituted by a phenyl group. Such a group is for example a phenylmethyl group, a 2-(phenyl)ethyl group, a 1-(phenyl)ethyl group, a phenylpropyl group or a phenylbutyl group.

When R₂ or R′₂ represents a piperidine ring, which is optionally substituted with an R₃ group, the positions of bonding on this ring can be done by any one of the members which can be substituted.

When R₂ or R′₂ represents a pyridine ring, which is optionally substituted with an R₈ group, the bonding positions and substitution positions can be done on any one of the carbons of the ring.

When R₂ or R′₂ represents a phenyl ring substituted with an R₇ group which is different to H, the relative positions of the substituents can be ortho, meta or para, with a preference for the para position.

“Addition salts” is to be understood as meaning addition salts which are obtained by a reaction of a compound of formula I containing at least one basic function in its non-salified form, with a mineral or organic acid. Preferably, they will be pharmaceutically acceptable addition salts.

Hydrochloric, hydrobromic, phosphoric and sulphuric acids are preferred amongst the mineral acids which are suitable for salifying a basic compound of formula I. Methanesulphonic, benzenesulphonic, toluenesulphonic, maleic, fumaric, oxalic, citric, tartaric, lactic and trifluoroacetic acids are preferred amongst the organic acids which are suitable for salifying a basic compound of formula I.

Those compounds in which R represents a phenylethyl group or a methyl group or an acetamide group, and those compounds in which one of the R₁ or R₂ substituents comprises a 5-imidazolyl or an “amidinyl” group in its structure, are preferred amongst the compounds according to the present invention, it being understood that “amidinyl” group means a group which contains the linked atoms:

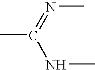

in its structure.

Thus, the “amidinyl” group includes amidine, 2-imidazolyl or 4,5-dihydro-2-imidazolyl groups, for example.

Those compounds in which W represents a chlorine atom, X represents a methyl group or a chlorine atom, Y represents a hydrogen atom or a chlorine atom, and Z represents a hydrogen atom, are also preferred amongst the compounds of the invention.

According to the invention, a general method of preparation of the compounds of the invention is recommended, which method comprises the steps consisting in:

1) allowing the benzenesulphonyl chloride of formula

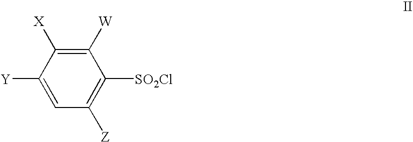

in which W, X, Y, Z are as defined above, to react with an amine of formula RNH₂ in which R represents a group as defined above, in a solvent such as dichloromethane and in the presence of an aprotic base such as triethylamine, to obtain a derivative of formula

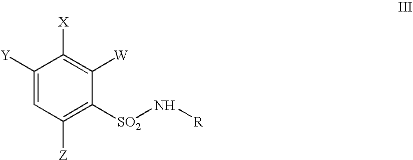

2) allowing the compound of formula III obtained above to react with the ethyl ester of N-(2-chloroacetyl)glycine

ClCH₂—CO—NH—CH₂—CO₂—C₂H₅ IV

in a solvent such as dimethylformamide and in the presence of a base such as potassium carbonate for example, to obtain a derivative of formula:

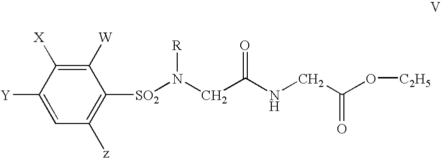

3) hydrolysing the ester bond of the compound of formula V, by the action of a strong base such as potassium hydroxide, in the presence of water and optionally a miscible organic solvent, to obtain an N-substituted glycine of formula VI:

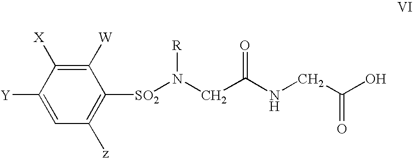

4) allowing the N-substituted glycine VI obtained above to react with a primary or secondary amine of formula

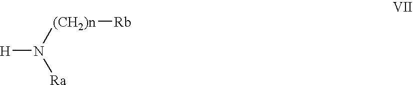

in which:

n represents 1, 2, 3 or 4,

Ra represents a hydrogen atom or a (CH₂)ₘR′b group in which m represents 1, 2, 3 or 4,

Rb and R′b independently each represent a hydrogen atom,

a

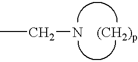

group, in which p represents 4, 5 or 6;

a

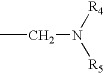

group, in which R₄ represents an amino-protecting group, COCH₃, COOCH₃, or a C₁-C₄ alkyl group, and R₅ represents a C₁-C₄ alkyl group which is optionally substituted with a phenyl group;

a

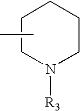

group, in which R₃ represents an amino-protecting group or a C₁-C₄alkyl group;

a

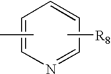

group, in which R₈ represents H, C₁-C₄ alkyl or NHRC in which Rc represents an amino-protecting group;

a

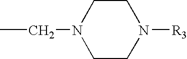

group, in which R₃ represents a C₁-C₄ alkyl group or an amino-protecting group;

a

—CH₂—O—R₆

group, in which R₆ represents H or CONHC₂H₅;

or a

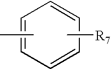

group, in which R₇ represents H, CN or CONHCH₃ in a solvent such as dichloromethane for example, in the presence of at least one coupling agent such as 1-(3-dimethylaminopropyl)-3-ethyl-carbodiimide (EDCI) or 1-hydroxy-7-azabenzotriazole (HOAT), in order to obtain the glycinamide of formula VIII

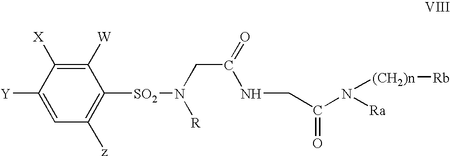

in which Ra, Rb and n keep the same meaning as above,

5) if necessary, the compound of formula VIII obtained above is allowed to react so as to replace the amino-protecting group(s) R₃ or R_{C} by a hydrogen atom, for example, if the R₃ group is a t-butyloxycarbonyl (Boc) group, by the action of trifluoroacetic acid in the presence of anisole and in a solvent, in order to obtain the compound of formula VIII in which the substituents keep the same meaning as above with the exception of R₃ and Rc which represent a hydrogen atom

6) and, or, if necessary, if the R₇ group is present and represents a cyano group, the compound of formula VIII is allowed to react:

ao) with ethylenediamine, in the presence of sulphur, in order to convert the R₇ group into a 4,5-dihydro-2-imidazolyl group; or successively:

a) with hydroxylamine, in a solvent such as DMSO in order to convert the R₇ group into an (amino)(hydroxyimino)methyl group,

b) then with acetic anhydride, in a solvent in order to convert the R₇ group into an (acetoxyimino)(amino)methyl group,

c) and then with hydrogen in the presence of a hydrogenation catalyst such as palladium on carbon, in a solvent such as methanol, in order to convert the R₇ group into an aminoiminomethyl group,

d) if necessary the compound thus obtained is allowed to react so as to replace the amino-protecting group(s) R₃ or R_{C} by a hydrogen atom and to thus obtain the compound of formula I

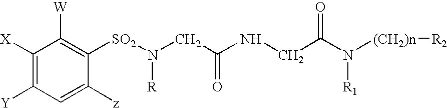

7) if necessary, the compound of formula I obtained above, when this compound comprises a basic function, is allowed to react with a mineral or organic acid in order to obtain the acid salt.

According to the invention, a method is also recommended of preparing the compounds of formula I in which at least one of the substituents R₁ and R₂ comprises a primary or secondary amine function (notably compounds of formula I comprising a group in which R₃ or R₄ or R₅ is a hydrogen atom), said method, known as “solid phase” method, consisting in:

a) fixing a diamine of general formula

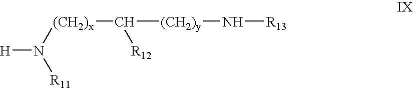

in which R₁₁ and R₁₂ independently each represent a hydrogen atom or a C₁-C₄ alkyl group, or together form a C₁-C₃ alkylene chain,

x represents 2 or 3,

y represents 0 or 1

R₁₃ represents an amino-protecting group, such as an Fmoc group for example,

onto a polystyrene resin which is functionalised with the aid of a chlorotrityl group represented by the formula

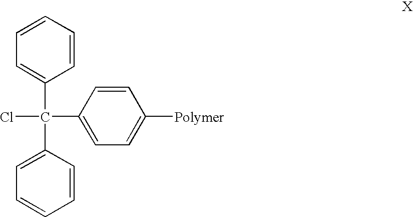

in which “polymer” represents the styrene polymer, said resin being abbreviated in the following as Res-Cl, in the presence of a tertiary amine and of a solvent of the diamine, in order to obtain the grafted resin of structure

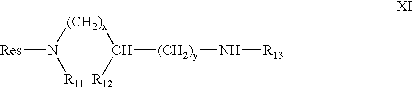

in which R₁₁, R₁₂, x, y and R₁₃ keep the same meaning as above,

b) deprotecting the amine function which is protected by the amino-protecting group R₁₃, for example in allowing the resin of formula XI to react with piperidine in the presence of a solvent if the R₁₃ group is an Fmoc group, so as to obtain the grafted resin of formula

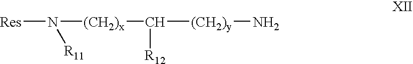

in which R₁₁, R₁₂, x and y keep the same meaning as above;

c) allowing the resin of formula XII to react with 2-nitrobenzenesulphonyl chloride in the presence of a solvent and of an aprotic base, such as diisopropylethylamine (DIPEA) for example, in order to obtain the resin of formula

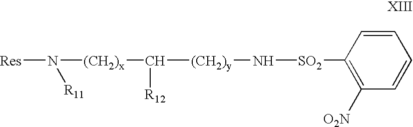

in which R₁₁, R₁₂, x and y keep the same meaning as above;

d) allowing the resin of formula XIII to react with an alcohol of general formula XIV

HO(CH₂)n—R_{b} XIV

in which

n represents 1, 2, 3 or 4 and Rb represents

a

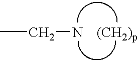

group, in which p represents 4, 5 or 6

a

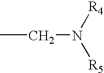

group, in which R₄ represents an amino-protecting group, COCH₃, COOCH₃, or a C₁-C₄ alkyl group and R₅ represents a C₁-C₄ alkyl group which is optionally substituted with a phenyl group;

a

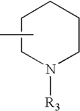

group, in which R₃ represents an amino-protecting group or a C₁-C₄ alkyl group,

a

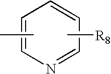

group, in which R₈ represents H, C₁-C₄ alkyl or NHRc in which Rc represents an amino-protecting group,

a

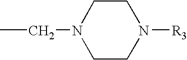

group, in which R₃ represents a C₁-C₄ alkyl group or an amino-protecting group,

in the presence of a solvent, of triphenylphosphine and of a coupling agent such as diisopropylazodicarboxylate (DIAD) in order to obtain the grafted resin of formula XV

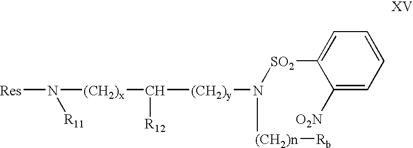

in which R₁₁, R₁₂, x, y, n and Rb keep the same meaning as above;

e) allowing the resin of formula XV to react with thiophenol in the presence of a solvent and of triethylamine so as to eliminate the 2-nitrobenzenesulphonyl group and to obtain the grafted resin of formula

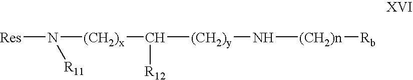

in which R₁₁, R₁₂, x, y, n and Rb keep the same meaning as above;

f) allowing the resin of formula XVI to react with the acid of formula VI obtained according to the steps 1 to 3 of the general method described above, in the presence of a solvent and of coupling agents such as diisopropylcarbodiimide (DIC) and hydroxybenzotriazole (HOBT), in order to form the amide bond and to obtain the resin of formula XVII:

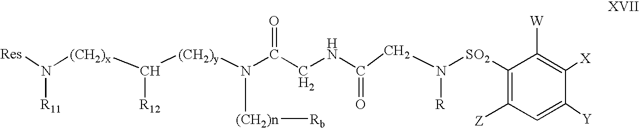

in which W, X, Y, Z, R₁₁, R₁₂, R_{b}, x, y and n keep the same meaning as above;

g) allowing the resin of formula XVII to react with trifluoroacetic acid in the presence of a solvent, so as to break the grafting bond on the resin and, simultaneously, to remove it if it exists, an amino-protecting group comprised in the Rb group and to thus obtain the compound of formula XVIII according to the invention, in the form of a salt with trifluoroacetic acid:

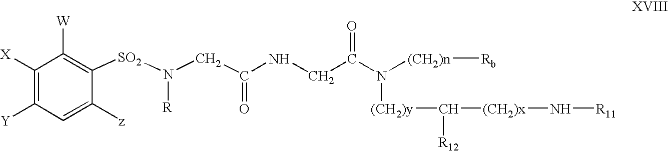

in which R, R₁₁, R₁₂, x, y and n keep the same meaning as above and R_{b} represents

a

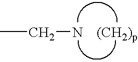

group, in which p represents 4, 5 or 6

a

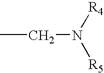

group, in which R₄ represents a hydrogen atom, COCH₃, COOCH₃, or a C₁-C₄ alkyl group and R₅ represents a C₁-C₄ alkyl group which is optionally substituted with a phenyl group;

a

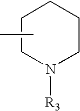

group, in which R₃ represents a hydrogen atom or a C₁-C₄ alkyl group,

a

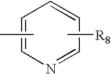

group, in which R₈ represents a hydrogen atom,

a C₁-C₄ alkyl group or NH₂ group, a

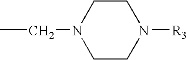

group, in which R₃ represents a hydrogen atom, or a C₁-C₄ alkyl group.

In a variant of the solid phase method described above, some compounds according to the invention can be prepared by carrying out the steps consisting in:

a) allowing a grafted resin of formula XII obtained according to step (b) of the method described above, to react with an acid of formula

HOOC—R_{b} XIX

in which R_{b} represents a N-Boc-piperidine group, in a solvent and in the presence of coupling agents such as diisopropylcarbodiimide and 1-hydroxybenzotriazole, in order to obtain the resin of formula XX

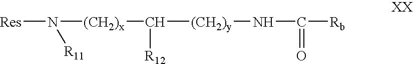

in which R₁₁, R₁₂, Rb, x and y keep the same meaning as in the starting compounds;

b) reducing the amide function of the grafted resin of formula XX by the action of borane-dimethylsulphide complex, in the presence of a solvent, in order to obtain the resin of formula XXI

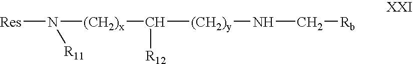

in which R₁₁, R₁₂, x, y and R_{b} keep the same meaning as above;

c) allowing the supported amine of formula XXI to react with the acid of formula VI obtained according to the steps 1 to 3 of the general method, under conditions analogous to those for carrying out step f of the solid phase method above and to thus obtain the resin of formula:

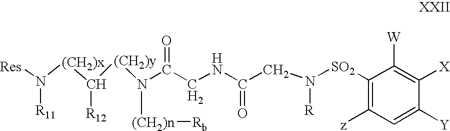

in which R₁₁, R₁₂, x, y, R_{b} keep the same meaning as above, and R represents a C₁-C₄ alkyl group, which is optionally substituted with a phenyl group,

d) allowing the resin thus obtained to react with trifluoroacetic acid, so as to break the grafting bond on the resin and to remove the amino-protecting group in order to obtain the compound of formula XXIII according to the invention, in the form of its salt with trifluoroacetic acid

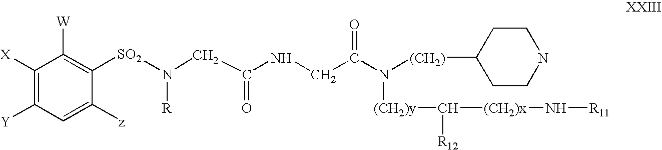

In the experimental part relating to the syntheses carried out in the solid phase, the amino-protecting groups as well as some solvents and some reagents shall be designated by abbreviations in a conventional manner:

Fmoc=9-fluorenylmethyloxycarbonyl

Boc=1,1-dimethylethoxycarbonyl

DIPEA=N,N-diisopropylethylamine

DIC=diisopropylcarbodiimide

DIAD=diisopropylazodicarboxylate

HOBT=1-hydroxybenzotriazole hydrate

DCM=dichloromethane

THF=tetrahydrofuran

DMF=dimethylformamide

The solid support (resin) is, unless indicated otherwise, a styrene polymer (PS) which is cross-linked with the aid of 1% of divinylbenzene and which is functionalised with a chlorotrityl group. This solid support enables fixing a substituted amine RNH₂ by forming the substituted resin:

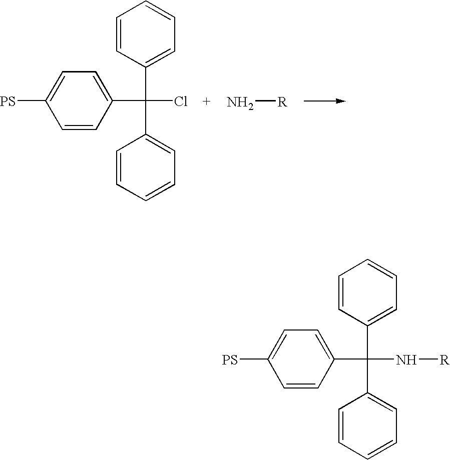

In order to simplify the text, the solid support will, in the following, be noted Res-, preceded by the position of substitution on R.

As an example, the compound of formula:

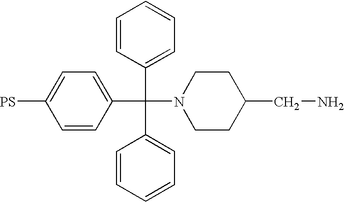

in which PS represents the polystyrene support will be designated: 4-(aminomethyl)-1-Res-piperidine.

In the descriptions of methods in relation to the syntheses in the solid phase, the agitation devices are always orbital movement agitators, without agitator inside the reaction vessel.

The identification and the purity of the novel compounds which are prepared in the solid phase are determined by means of an analysis by LC/MS coupling (liquid phase chromatography coupled with a mass spectrometer). Unless indicated otherwise, the chromatography is carried out on a Hewlett Packard HP1100 chain equipped with a 50×4.6 mm column packed with stationary phase of the C18 grafted silica type, 3.5 or 5 μm (for example referenced SYMMETRY from WATERS). The column is thermostated at 30° C. The mobile phase, regulated on a flow rate of 0.4 or 1 ml/min, is a gradient of the following solvents A and B:

A: distilled water containing 0.05% of trifluoroacetic acid

B: acetonitrile containing 0.05% of trifluoroacetic acid

The various gradient conditions employed for the analyses are the following (the values indicated in the Table are the proportion in % of solvent B in the mixture A+B).

| | | | | | | | | | | flow rate |
|---|---|---|---|---|---|---|---|---|---|---|
| T (min) | 0 | 5 | 6 | 7 | 8 | 9 | 10 | 12 | column | (ml/min) |
| Grad. A | 25 | 90 | 90 | 25 | | | 25 | | I | 1 |
| Grad. B | 30 | 90 | 90 | 30 | | | 30 | | I | 1 |
| Grad. C | 30 | 90 | 90 | 30 | | | 30 | | II | 1 |
| Grad. D | 30 | | | 30 | 90 | 30 | | 30 | III | 0.4 |

Column I: 50 × 4.6 mm column, C₁₈ grafted silica 3.5 μm (SYMMETRY/WATERS)
Column II: 50 × 4.6 mm column, C₁₈ grafted silica 5 μm (SYMMETRY/WATERS)
Column III: 50 × 3 mm column, C₁₈ ODB grafted silica 3 μm (UPTISPHERE)

The mass spectrometer is a PERKIN.ELMER SCIEX API 150 MCA apparatus with detection by positive ionisation APCI⁺.

The analytical result, indicated “LC/MS” after each Preparation Or Example, mentions the conditions of the analysis (Grad. X) and the retention time of the compound expressed in minutes and fractions of minutes.

The invention will be better understood upon reading the Examples of preparation as well as the results of pharmacological tests made with compounds according to the invention. The aim of these non-limiting examples is only for illustrating the invention and will in no case limit the scope thereof.

Amongst the abbreviations used in the following descriptions, mM signifies millimole (10³ mole).

Preparation I

2,4-dichloro-3-methyl-N-(2-phenylethyl)-benzenesulphonamide

A solution of 59.6 g (0.23 mole) of 2,4-dichloro-3-methylbenzenesulphonyl chloride is prepared in 500 ml of dichloromethane and 25.2 g (0.25 mole) of triethylamine are added, and then 31.4 ml (0.25 mole) of 2-phenylethylamine, dropwise. The reaction mixture is maintained under agitation for 15 hours at ambient temperature, and then washed successively with a normal hydrochloric acid solution, a saturated sodium bicarbonate solution and then with water. The organic phase is dried over magnesium sulphate, and then concentrated under reduced pressure. The residue obtained is crystallised in petroleum ether. 63.9 g of the product sought after are thus obtained in the form of a white solid (yield=81%).

M.Pt.=75° C.

Preparation II

N-[2-[[(2,4-dichlor-3-methylphenyl)sulphonyl](2-phenylethyl)amin]acetyl]glycine, Ethyl Ester

24.05 g (0.174 M) of potassium carbonate are added, and then 31.23 g (0.174 M) of the ethyl ester of N-chloroacetylglycine are added to a solution of 47 g (0.139 M) of the compound obtained according to preparation I in 300 ml of DMF. The reaction mixture is agitated at ambient temperature for 28 hours and then poured onto water.

The precipitate formed is separated off by filtration and then taken up into solution in ethyl acetate. This organic phase is washed with a normal hydrochloric acid solution, and then with a saturated sodium bicarbonate solution and then with water. After drying over sodium sulphate and then concentration under reduced pressure, a solid is obtained which is recrystallised from isopropyl alcohol. 45.4 g of the product sought after are thus obtained in the form of a white solid (yield=67%).

M.Pt.=100° C.

Preparation III

N-[2-[[(2,4-dichloro-3-methylphenyl)sulphonyl](2-phenylethyl)amino]acetyl]glycine

A solution of 48.7 g (99.9 mM) of the ester obtained according to preparation II is prepared in 150 ml of dioxane, 150 ml of a normal sodium hydroxide solution are added and the mixture is then agitated at 50° C. for 3 hours. The reaction medium is then concentrated under reduced pressure and the residue is taken up with water and acidified with the aid of a normal hydrochloric acid solution. The mixture is extracted with ethyl acetate, the organic phase obtained is washed with water, dried and then concentrated under reduced pressure. The residue is crystallised in petroleum ether. 37 g of the product sought after are thus obtained in the form of a fine white solid (yield: 81%).

M.Pt.=110° C.

Preparation IV

[3-[[(4-cyanophenyl)methyl]amino]propyl]carbamic Acid, 1,1-dimethylethyl Ester

A solution of 2.61 g (15 mM) of the 1,1-dimethylethyl ester of (3-aminopropyl)carbamic acid is prepared in 10 ml of ethanol and 1 g (5.1 mM) of 4-(bromomethyl)benzonitrile in suspension in 10 ml of ethanol is added. The reaction mixture is agitated under reflux in a solvent for 18 hours and then concentrated under reduced pressure. The residue is purified by chromatography on silica gel in eluting with the aid of a dichloromethane/methanol/aqueous ammonia mixture (98/2/0.2; v/v/v). 1.3 g of the product sought after are thus obtained in the form of a white solid (yield=88%).

M.Pt.=64° C.

Preparation V

[4-[[(4-cyanophenyl)methyl]amino]butyl]carbamic Acid, 1,1-dimethylethyl Ester

In performing analogously to preparation IV, starting with the 1,1-dimethylethyl ester of (4-aminobutyl)carbamic acid, the product sought after is obtained in the form of a white solid (yield=87%).

M.Pt.=48-50° C.

Preparation VI [4-[[(4-cyanophenyl)methyl][2-[[2-[[(2,4-dichloro-3-methylphenyl)sulphonyl](2-phenylethyl)amino]acetyl]amino]acetyl]amino]-butyl]carbamic acid, 1,1-dimethylethyl Ester

A solution of 0.4 g (0.871 mM) of the acid obtained according to preparation III is prepared in 20 ml of dichloromethane, and 0.18 g (0.958 mM) of 1-(3-dimethylaminopropyl)-3-ethyl-carbodiimide hydrochloride (EDCI), and then 0.13 g (0.958 mM) of 1-hydroxy-7-azabenzotriazole (HOAT) are added. The reaction mixture is agitated at ambient temperature for 20 minutes and 0.1 g (1 mM) of triethylamine and 0.29 g (0.958 mM) of the amine obtained according to preparation V are then added. The mixture is agitated for 48 hours at ambient temperature and is then poured onto water. After separation and removal of the aqueous phase, the organic phase is dried over magnesium sulphate and then concentrated under reduced pressure. The residue is purified by chromatography on silica gel in eluting with the aid of a dichloromethane/methanol mixture (8/2; v/v). 0.48 g of the product sought after is thus obtained in the form of a white amorphous solid (yield=74%).

M.Pt.=87%

Preparation VII

[4-[[[4-[amino(hydroxyimino)methyl]phenyl]methyl][2-[[2-[[(2,4-dichloro-3-methylphenyl)sulphonyl](2-phenylethyl) amino]acetyl]amino]-acetyl]amino]-butyl]carbamic Acid, 1,1-dimethylethyl Ester

A solution of 0.45 g (0.604 mM) of the compound obtained according to preparation VI is prepared in 10 ml of DMSO and 0.15 g (2.1 mM) of hydroxylamine hydrochloride and 0.427 g (4.2 mM) of triethylamine are added. The reaction mixture is agitated for 24 hours at ambient temperature. 0.15 g of hydroxylamine hydrochloride and 0.427 g of triethylamine are added once more and the reaction mixture is agitated further for 24 hours. The mixture is then poured onto water and a precipitate is formed which is filtered off, rinsed with water and dried under reduced pressure. 0.43 g of the compound sought after is thus obtained in the form of a white solid (yield=91%).

M.Pt.=102° C.

Preparation VIII

[4-[[[4-[[(acetyloxy)imino](amino)methyl]phenyl]methyl][2-[[2-[[(2,4-dichloro-3-methylphenyl)sulphonyl](2-phenylethyl)-amino]acetyl]amino]acetyl]amino]butyl]carbamic Acid, 1,1-dimethylethyl Ester

175 mg (1.6 mM) of acetic anhydride are added to a solution of 0.42 g (0.54 mM) of the compound obtained according to preparation VII in 20 ml of dichloromethane. After 20 hours under agitation at ambient temperature (20 to 25° C.) the reaction mixture is washed with the aid of a saturated sodium bicarbonate solution and then with water. The organic phase is dried over magnesium sulphate and concentrated under reduced pressure. 0.43 g of the product sought after is thus obtained in the form of a white amorphous solid (yield: 97%).

M.Pt.=100° C.

Preparation IX

[4-[[[4-(aminoiminomethyl)phenyl]methyl][2-[[2-[[(2,4-dichloro-3-methylphenyl)sulph nyl](2-phenylethyl)amin]acetyl]amin]acetyl]-amin]butyl]carbamic Acid, 1,1-dimethylethyl Ester

A solution of 0.39 g (0.476 mM) of the compound obtained according to preparation VIII is prepared in 30 ml of methanol and 40 mg of platinum on carbon (5% Pt) is added. The mixture is agitated under a hydrogen atmosphere, at atmospheric pressure and at ambient temperature for 4 hours. The catalyst is removed by filtration and the filtrate is concentrated under reduced pressure. The residue is purified by chromatography on NH₂ grafted silica gel (Lichroprep NH₂-40-63 μm), in eluting with the aid of a dichloromethane/methanol mixture (96/4; v/v). 0.37 g of the product sought after is thus obtained in the form of a white solid (yield=100%).

M.Pt.=122° C.

### EXAMPLE 1

N-[2-[(4-aminobutyl)[[4-(aminoiminomethyl)phenyl]methyl]-amino]-2-oxoethyl]-2-[[(2,4-dichloro-3-methylphenyl)sulphonyl]-(2-phenylethyl)amino]acetamide, Bis Trifluoroacetate

A mixture of 0.36 g (0.473 mM) of the compound obtained according to preparation 1× and 0.051 g (0.473 mM) of anisole is prepared and 1.5 ml of trifluoroacetic acid are added. The solution obtained is agitated for 4 hours at ambient temperature, and then concentrated under reduced pressure. 5 ml of toluene are then added to the residue and concentration is effected once more under reduced pressure so as to remove the excess of trifluoroacetic acid. The solid residue is triturated with diethyl ether and the liquid phase is removed. The residual solid product is taken up into solution in 10 ml of distilled water and the solution is filtered and lyophilised. 0.28 g of the product sought after is thus obtained in the form of a white, fine, light solid (yield=67%).

M.Pt.=123° C.

Preparation X

[3-[[(4-cyanophenyl)methyl][2-[[2-[[(2,4-dichloro-3-methylphenyl)sulphonyl](phenylethyl)amino]acetyl]amino]acetyl]amino]-propyl]carbamic Acid, 1,1-dimethylethyl Ester

In performing analogously to preparation VI, starting with the compound obtained according to preparation IV, the product sought after is obtained in the form of a white amorphous solid (yield=45%).

M.Pt.=80° C.

Preparation XI

[3-[[[4-amino(hydroxyimino)methyl]phenyl]methyl][2-[[2-[[(2,4-dichloro-3-methylphenyl)sulphonyl](phenylethyl)-amino]acetyl]amino]-acetyl]amino]propyl]carbamic Acid, 1,1-dimethylethyl Ester

In performing analogously to preparation VII, starting with the compound obtained according to preparation X, the product sought after is obtained in the form of a white solid (yield=96%).

M.Pt.=112° C.

Preparation XII

[3-[[[4-[[(acetyloxy)imino)methyl]phenyl]methyl][2-[[2-[[(2,4-dichloro-3-methylphenyl)sulphonyl](2-phenylethyl)amino]-acetyl]amino]-acetyl]amino]propyl]carbamic Acid, 1,1-dimethylethyl Ester

In performing analogously to preparation VIII, starting with the compound obtained according to preparation XI, the product sought after is obtained in the form of a white solid (yield=93%).

M.Pt.=92° C.

Preparation XIII

[3-[[[4-(aminoiminomethyl)phenyl]methyl][2-[[2-[[(2,4-dichloro-3-methylphenyl)sulphonyl](2-phenylethyl)amino]acetyl]-amino]acetyl]-amino]propyl]carbamic acid, 1,1-dimethylethyl Ester

In performing analogously to preparation IX, starting with the compound obtained according to preparation XII, the product sought after is obtained in the form of a white solid (yield=69%).

M.Pt.=106° C.

### EXAMPLE 2

N-[2-[(3-aminopropyl)[[4-(aminoiminomethyl)phenyl]methyl]-amino]-2-oxoethyl]-2-[[(2,4-dichloro-3-methylphenyl)sulphonyl]-(2-phenylethyl)amino]acetamide, Bis Trifluoroacetate

In performing analogously to Example 1, starting with the compound obtained according to preparation XIII, the product sought after is obtained in the form of a white, fine and light solid (yield=93%).

M.Pt.=128° C.

Preparation XIV

4-[[[3-(1-pyrrolidinyl)propyl]amino]methyl]benzonitrile

A solution of 1.96 g (15.3 mM) of 1-(3-aminopropyl)pyrrolidine is prepared in 25 ml of toluene and 2 g (15.3 mM) of 4-cyanobenzaldehyde are added. The solution is heated under reflux under agitation and the water formed from the reaction is removed by means of a Dean-Stark apparatus.

The reaction lasts for about 6 hours. The solvent is then removed under reduced pressure and the residue is taken up into solution in 25 ml of methanol. 0.58 g (15.3 mM) of sodium borohydride is added and the reaction medium is maintained under agitation for 20 hours at ambient temperature.

The mixture is then concentrated under reduced pressure, the residue is taken up in dichloromethane and the organic phase obtained is washed with water twice, and then dried over magnesium sulphate and concentrated under reduced pressure. The product sought after is thus obtained in the form of an orange oil (yield=95%).

NMR (¹H, 300 MHz, CDCl₃) 7.78 (d, 2H); 7.52 (d, 2H); 3.74 (s, 2H); 2.49 (m, 2H); 2.35 (m, 6H); 1.62 (m, 6H).

Preparation XV

N-[2-[[(4-cyanophenyl)methyl][3-(1-pyrrolidinyl)propyl]amino]-2-oxoethyl]-2-[[(2,4-dichloro-3-methylphenyl)sulphonyl](2-phenylethyl)-amino]acetamide

In performing analogously to preparation VI, starting with the compound obtained according to preparation XIV, the product sought after is obtained in the form of a white amorphous powder (yield=69%).

M.Pt.=88° C.

### EXAMPLE 3

N-[2-[[[4-[amino(hydroxyimino)methyl]phenyl]methyl][3-(1-pyrrolidinyl)propyl]amino]-2-oxoethyl]-2-[[(2,4-dichloro-3-methylphenyl)sulphonyl](2-phenylethyl)amino]acetamide

In performing analogously to preparation VII, starting with the compound obtained according to preparation XV, the product sought after is obtained in the form of a white solid (yield=90%).

M.Pt.=92° C.

### EXAMPLE 4

N-[2-[[[4-[[(acetyloxy)imino](amino)methyl]phenyl]methyl][3-(1-pyrrolidinyl)propyl]amino]-2-oxoethyl]-2-[[(2,4-dichloro-3-methylphenyl)sulphonyl](2-phenylethyl)amino]acetamide

In performing analogously to preparation VIII, starting with the compound obtained according to Example 3, the product sought after is obtained in the form of a white solid (yield=79%).

M.Pt.=90° C.

### EXAMPLE 5

N-[2-[[[4-(aminoiminomethyl)phenyl]methyl][3-(1-pyrrolidinyl)propyl]amino]-2-oxoethyl]-2-[[(2,4-dichloro-3-methylphenyl)sulphonyl](2-phenylethyl)amino]acetamide

In performing analogously to preparation IX, starting with the compound obtained according to Example 4, the product sought after is obtained in the form of a white solid (yield=39%).

M.Pt.=105° C.

### EXAMPLE 6

N-[2-[[[4-(aminoiminomethyl)phenyl]methyl][3-(1-pyrrolidinyl)propyl]amino]-2-oxoethyl]-2-[[(2,4-dichloro-3-methylphenyl)sulphonyl](2-phenylethyl)amino]acetamide, Dihydrochloride

A solution of 80 mg (0.11 mM) of the compound obtained according to Example 5 is prepared in 5 ml of methanol and 1 ml of a saturated solution of hydrogen chloride in ethyl ether is added. The mixture is agitated for 1 hour and then concentrated under reduced pressure. The solid residue is taken up into solution in 5 cm³ of distilled water, the solution is filtered and then lyophilised. 88 mg of the product sought after are thus obtained in the form of a fine white solid (yield=100%).

M.Pt.=145° C.

Preparation XVI

4-[[[2-(1-pyrr lidinyl)ethyl]amin]methyl]benzonitrile

In performing analogously to preparation XIV, starting with 1-(2-aminoethyl)pyrrolidine, the product sought after is obtained in the form of a yellow oil (yield=77%).

NMR (¹H, 300 MHz): 7.7 (d, 2H); 7.5 (d, 2H); 3.77 (s, 2H); 2.50 (m, 4H); 1.66 (m, 4H)

Preparation XVII

N-[2-[[(4-cyanophenyl)methyl][2-(1-pyrrolidinyl)ethyl]amino]-2-oxo-ethyl]-2-[[(2,4-dichloro-3-methylphenyl)sulphonyl](2-phenylethyl)amino]acetamide

In performing analogously to preparation VI, starting with the compound obtained according to preparation XVI, the product sought after is obtained in the form of a white solid (yield=77%).

M.Pt.=65° C.

### EXAMPLE 7

N-[2-[[[4-[amino(hydroxyimino)methyl]phenyl]methyl][2-(1-pyrrolidinyl)ethyl]amino]-2-oxoethyl]-2-[[(2,4-dichloro-3-methylphenyl)sulphonyl](2-phenylethyl)amino]acetamide

In performing analogously to preparation VII, starting with the compound obtained according to preparation XVII, the product sought after is obtained in the form of a white solid (yield=97%).

M.Pt.=85° C.

### EXAMPLE 8

N-[2-[[[4-[[(acetyloxy)imino](amino)methyl]phenyl]methyl][2-(1-pyrrolidinyl)ethyl]amino]-2-oxoethyl]-2-[[(2,4-dichloro-3-methylphenyl)sulphonyl](2-phenylethyl)amino]acetamide

In performing analogously to preparation VIII, starting with the compound obtained according to Example 7, the product sought after is obtained in the form of a white solid (yield=91%).

M.Pt.=82° C.

### EXAMPLE 9

N-[2-[[[4-(aminoiminomethyl)phenyl]methyl][2-(1-pyrrolidinyl)ethyl]-amino]-2-oxoethyl]-2-[[(2,4-dichloro-3-methylphenyl)sulphonyl](2-phenylethyl)amino]acetamide

In performing analogously to preparation IX, starting with the compound obtained according to Example 8, the product sought after is obtained in the form of a white solid (yield=52%).

M.Pt.=106° C.

### EXAMPLE 10

N-[2-[[[4-(aminoiminomethyl)phenyl]methyl][2-(1-pyrrolidinyl)ethyl]amino]-2-oxoethyl]-2-[[(2,4-dichloro-3-methylphenyl)sulphonyl](2-phenylethyl)amino]acetamide, Dihydrochloride

In performing analogously to Example 6, starting with the compound obtained according to Example 9, the product sought after is obtained in the form of a fine white powder (yield=94%).

M.Pt.=140° C.

Preparation XVIII

4-[[[4-(1-pyrrolidinyl)butyl]amino]methyl]benzonitrile

In performing analogously to preparation XIV, starting with 1-(4-aminobutyl)pyrrolidine, the product sought after is obtained in the form of an orange oil (yield=81%).

NMR (¹H, 300 MHz, CDCl₃): 7.77 (d, 2H); 7.51 (d, 2H); 3.75 (s, 2H); 2.38 (m, 8H); 1.67 (m, 4H); 1.44 (m, 4H)

Preparation XIX

N-[2-[[(4-cyanophenyl)methyl][4-(1-pyrrolidinyl)butyl]amino]-2-oxo-ethyl]-2-[[(2,4-dichloro-3-methylphenyl)sulphonyl](2-phenylethyl)amino]acetamide

In performing analogously to preparation VI, starting with the compound obtained according to preparation XVIII, the product sought after is obtained in the form of an off-white amorphous solid (yield=62%).

M.Pt.=70° C.

### EXAMPLE 11

N-[2-[[[4-[amino(hydroxyimino)methyl]phenyl]methyl][4-(1-pyrrolidinyl)butyl]amino]-2-oxoethyl]-2-[[(2,4-dichloro-3-methylphenyl)sulphonyl](2-phenylethyl)amino]acetamide

In performing analogously to preparation VII, starting with the compound obtained according to preparation XIX, the product sought after is obtained in the form of a white solid (yield=96%).

M.Pt.=92° C.

### EXAMPLE 12

N-[2-[[[4-[[(acetyloxy)imino](amino)methyl]phenyl]methyl][4-(1-pyrrolidinyl)butyl]amino]-2-oxoethyl]-2-[[(2,4-dichloro-3-methylphenyl)sulphonyl](2-phenylethyl)amino]acetamide

In performing analogously to preparation VIII, starting with the compound obtained according to Example 11, the product sought after is obtained in the form of a white solid (yield=90%).

M.Pt.=88° C.

### EXAMPLE 13

N-[2-[[[4-(aminoiminomethyl)phenyl]methyl][4-(1-pyrrolidinyl)butyl]amino]-2-oxoethyl]-2-[[(2,4-dichloro-3-methylphenyl)sulphonyl](2-phenylethyl)amino]acetamide

In performing analogously to preparation IX, starting with the compound obtained according to Example 12, the product sought after is obtained in the form of a white amorphous solid (yield=40%).

M.Pt.=155° C.

### EXAMPLE 14

N-[2-[[[4-(aminoiminomethyl)phenyl]methyl][4-(1-pyrrolidinyl)butyl]amino]-2-oxoethyl]-2-[[(2,4-dichloro-3-methylphenyl)sulphonyl](2-phenylethyl)amino]acetamide, dihydrochloride

In performing analogously to Example 6, starting with the compound obtained according to Example 13, the product sought after is obtained in the form of a white, fine, light solid (yield=100%).

M.Pt.=155° C.

Preparation XX

4-[[[4-(dimethylamino)butyl]amino]methyl]benzonitrile

In performing analogously to preparation XIV, starting with N,N-dimethyl-1,4-butanediamine, the product sought after is obtained in the form of a yellow oil (yield=77%).

NMR (¹H, 300 MHz, CDCl₃): 7.76 (d, 2H); 7.52 (d, 2H); 3.74 (s, 2H); 2.49 (m, 2H); 2.14 (m, 2H); 2.08 (s, 6H); 1.39 (m, 4H).

Preparation XXI

N-[2-[[(4-cyanophenyl)methyl][4-(dimethylamino)butyl]amino]-2-oxo-ethyl]-2-[[(2,4-dichloro-3-methylphenyl)sulphonyl](2-phenyl ethyl)amino]-acetamide

In performing analogously to preparation VI, starting with the compound obtained according to preparation XX, the product sought after is obtained in the form of a white amorphous solid (yield=68%).

M.Pt.=60° C.

### EXAMPLE 15

N-[2-[[[4-[amino(hydroxyimino)methyl]phenyl]methyl][4-(dimethylamino)butyl]amino]-2-oxoethyl]-2-[[(2,4-dichloro-3-methylphenyl)-sulphonyl](2-phenylethyl)amino]acetamide

In performing analogously to preparation VII, starting with the compound obtained according to preparation XXI, the product sought after is obtained in the form of a white solid (yield=93%).

M.Pt.=92° C.

### EXAMPLE 16

N-[2-[[[4-[[(acetyloxy)imino](amino)methyl]phenyl]methyl][4-(dimethylamino)butyl]amino]-2-oxoethyl]-2-[[(2,4-dichloro-3-methyl-phenyl)sulphonyl](2-phenylethyl)amino]acetamide

In performing analogously to preparation VIII, starting with the compound obtained according to Example 15, the product sought after is obtained in the form of a white amorphous solid (yield=100%).

M.Pt.=55° C.

### EXAMPLE 17

N-[2-[[[4-(aminoimin methyl)phenyl]methyl][4-(dimethylamino)butyl]-amino]-2-oxoethyl]-2-[[(2,4-dichloro-3-methylphenyl)sulphonyl](2-phenylethyl)amino]acetamide

In performing analogously to preparation IX, starting with the compound obtained according to Example 16, the product sought after is obtained in the form of a white amorphous solid (yield=83%).

M.Pt.=78° C.

### EXAMPLE 18

N-[2-[[[4-(aminoiminomethyl)phenyl]methyl][4-(dimethylamino)butyl]amino]-2-oxoethyl]-2-[[(2,4-dichloro-3-methylphenyl)sulphonyl](2-phenylethyl)amino]acetamide, dihydrochloride

In performing analogously to Example 6, starting with the compound obtained according to Example 17, the product sought after is obtained in the form of a white, fine, light solid (yield=91%).

M.Pt.=148° C.

Preparation XXII

4-[[[3-(dimethylamino)propyl]amino]methyl]benzonitrile

In performing analogously to preparation XIV, starting with N,N-dimethyl-1,3-propanediamine, the product sought after is obtained in the form of an orange oil (yield=40%).

NMR (¹H, 300 MHz, DMSO): 7.91 (d, 2H); 7.53 (d, 2H); 3.74 (s, 2H); 3.3 (m, 1H); 2.48 (t, 2H); 2.21 (t, 2H); 2.08 (s, 6H); 1.53 (m, 2H).

Preparation XXIII

N-[2-[[(4-cyanophenyl)methyl][3-(dimethylamino)propyl]amino]-2-oxo-ethyl]-2-[[(2,4-dichloro-3-methylphenyl)sulphonyl](2-phenylethyl)amino]acetamide

In performing analogously to preparation VI, starting with the compound obtained according to preparation XXII, the product sought after is obtained in the form of a white solid (yield=51%).

M.Pt.=70° C.

### EXAMPLE 19

N-[2-[[[4-[amino(hydroxyimino)methyl]phenyl]methyl][3-(dimethylamino)propyl]amino]-2-oxoethyl]-2-[[(2,4-dichloro-3-methylphenyl)sulphonyl](2-phenylethyl)amino]acetamide

In performing analogously to preparation VII, starting with the compound obtained according to preparation XXIII, the product sought after is obtained in the form of a white solid (yield=98%).

M.Pt.=56-58° C.

### EXAMPLE 20

N-[2-[[[4-[amino(hydroxyimino)methyl]phenyl]methyl][3-(dimethylamino)propyl]amino]-2-oxoethyl]-2-[[(2,4-dichloro-3-methylphenyl)sulphonyl](2-phenylethyl)amino]acetamide, Dihydrochloride

In performing analogously to Example 6, starting with the compound obtained according to Example 19, the product sought after is obtained in the form of a fine white solid (yield=98%).

M.Pt.=142° C.

### EXAMPLE 21

N-[2-[[[4-[[(acetyloxy)imino](amino)methyl]phenyl]methyl][3-(dimethylamino)propyl]amino]-2-oxoethyl]-2-[[(2,4-dichloro-3-methyl-phenyl)sulphonyl](2-phenylethyl)amino]acetamide

In performing analogously to preparation VIII, starting with the compound obtained according to Example 19, the product sought after is obtained in the form of a white solid (yield=71%).

M.Pt.=90° C.

### EXAMPLE 22

N-[2-[[[4-(aminoiminomethyl)phenyl]methyl][3-(dimethylamino)propyl]amino]-2-oxoethyl]-2-[[(2,4-dichloro-3-methylphenyl)sulphonyl]-(2-phenylethyl)amino]acetamide

In performing analogously to preparation IX, starting with the compound obtained according to Example 21, the product sought after is obtained in the form of a white amorphous powder (yield=73%).

M.Pt.=114° C.

### EXAMPLE 23

N-[2-[[[4-(aminoiminomethyl)phenyl]methyl][3-(dimethylamin)propyl]amino]-2-oxoethyl]-2-[[(2,4-dichloro-3-methylphenyl)sulphonyl](2-phenylethyl)amino]acetamide, Dihydrochloride

In performing analogously to Example 6, starting with the compound obtained according to Example 22, the product sought after is obtained in the form of a fine white solid (yield=98%).

M.Pt.=157° C.

Preparation XXIV

4-[[[(4-cyanophenyl)methyl]amino]methyl]-1-piperidine-carboxylic Acid, 1,1-dimethylethyl Ester

In performing analogously to preparation XIV, starting with the t-butyl ester of 4-(aminomethyl)-1-piperidinecarboxylic acid, the product sought after is obtained in the form of a yellow oil (yield=88%).

NMR (¹H, 300 MHz, DMSO): 7.76 (d, 2H); 7.52 (d, 2H); 3.90 (d, 2H); 3.74 (s, 2H); 2.66 (m, 1H); 2.30 (d, 2H); 1.68 (d, 2H); 1.54 (m, 2H); 1.37 (s, 9H); 0.95 (m, 2H).

Preparation XXV

4-[[[(4-cyanophenyl)methyl][2-[[2-[[(2,4-dichloro-3-methylphenyl)sulphonyl](2-phenylethyl)amino]acetyl]amino]acetyl]-amino]methyl]-1-piperidine Carboxylic Acid, 1,1-dimethylethyl Ester

In performing analogously to preparation VI, starting with the compound obtained according to preparation XXIV, the product sought after is obtained in the form of a viscous oil (yield=84%).

NMR (¹H, 300 MHz, DMSO): 8.88 (m, 1H); 8.54 (m, 2H); 8.43 (d, 1H); 8.22 (d, 1H); 8.08 (d, 2H); 7.83 (m, 5H); 5.52 (s, 1H); 5.37 (s, 1H); 4.82 (d, 2H); 4.75 (d, 1H); 4.58 (m, 3H); 4.14 (m, 2H); 3.98 (m, 2H); 3.86 (d, 2H); 3.40 (m, 3H); 3.05 and 2.96 (2s, 3H); 2.22 (m, 2H); 2.045 (d, 9H); 1.68 (m, 2H).

Preparation XXVI

4-[[[[4-[amin(hydr xyimino)methyl]phenyl]methyl][2-[[2-[[(2,4-dichloro-3-methylphenyl)sulphnyl](2-phenylethyl)amino]acetyl]amino]-acetyl]amino]methyl]-1-piperidinecarboxylic Acid, 1,1-dimethylethyl Ester

In performing analogously to preparation VII, starting with the compound obtained according to preparation XXV, the product sought after is obtained in the form of a beige amorphous solid (yield=84%).

M.Pt.=100° C.

Preparation XXVII

4-[[[[4-[[(acetyloxy)imino](amino)methyl]phenyl]methyl][2-[[2-[[(2,4-dichloro-3-methylphenyl)sulphonyl](2-phenylethyl)amino]acetyl]amino]-acetyl]amino]methyl]-1-piperidinecarboxylic Acid, 1,1-dimethylethyl Ester

In performing analogously to preparation VIII, starting with the compound obtained according to preparation XXVI, the product sought after is obtained in the form of a white solid (yield=89%).

M.Pt.=110° C.

Preparation XXVIII

4-[[[[4-(aminoiminomethyl)phenyl]methyl][2-[[2-[[(2,4-dichloro-3-methylphenyl)sulphonyl](2-phenylethyl)amino]acetyl]amino]-acetyl]-amino]methyl]-1-piperidinecarboxylic Acid, 1,1-dimethylethyl Ester

In performing analogously to preparation IX, starting with the compound obtained according to preparation XXVII, the product sought after is obtained in the form of a white solid (yield=98%).

M.Pt.=140° C.

### EXAMPLE 24

N-[2-[[[4-(aminoiminomethyl)phenyl]methyl](4-piperidinylethyl)amino]-2-oxoethyl]-2-[[(2,4-dichloro-3-methylphenyl)sulphonyl](2-phenylethyl)-amino]acetamide, bis Trifluoroacetate

In performing analogously to Example 1, starting with the compound obtained according to preparation X)VIII, the product sought after is obtained in the form of a white solid (yield=88%).

M.Pt.=130° C.

Preparation XXIX

4-[[[(2,4-dinitrophenyl)sulphonyl]amino]methyl]-1-piperidinecarboxylic Acid, 1,1-dimethylethyl Ester

A solution of 5.36 g (25 mM) of the 1,1-dimethylethyl ester of 4-(aminomethyl)-1-piperidinecarboxylic acid is prepared in 60 ml of dichloromethane and a solution of 6.66 g (25 mM) of 2,4-dinitrobenzenesulphonyl chloride in 40 ml of dichloromethane, and then 2.52 g (25 mM) of pyridine are added. The reaction mixture is agitated for 18 hours at ambient temperature and then washed successively with a 0.1N hydrochloric acid solution, a saturated sodium bicarbonate solution and with pure water. After drying over sodium sulphate, the organic phase is concentrated under reduced pressure and the residue is purified by chromatography on silica gel in eluting with the aid of a cyclohexane/ethyl acetate mixture (6/4; v/v). 6.9 g of the product sought after are thus obtained in the form of a yellow solid (yield=62%).

M.Pt.=148° C.

Preparation XXX

4-[[[4-[[(1,1-dimethylethoxy)carbonyl]amino]butyl]amino]methyl]-1-piperidinecarboxylic Acid, 1,1-dimethylethyl Ester, Hydrochloride

a) a solution of 1.33 g (3 mM) of the compound obtained according to preparation XXIX is prepared in 20 ml of tetrahydrofuran. 1.57 g (6 mM) of triphenylphosphine, a solution of 1.13 g (6 mM) of the 1,1-dimethylethyl ester of (4-hydroxybutyl)carbamic acid in 20 ml of toluene and then 1 g (6 mM) of diethylazodicarboxylate are added. The mixture is agitated for 4 hours at ambient temperature. 10 g of silica gel for chromatography are then added before concentrating under reduced pressure. The powdery residue is then subjected to a preparative chromatography on silica gel in eluting with the aid of an ethyl acetate/hexane mixture (4/6; v/v). 1.86 g of the 1,1-dimethylethyl ester of 4-[[[4-[[(1,1-dimethylethoxy)carbonyl]amino]butyl][(2,4-dinitrophenyl)sulphonyl]amino]methyl]-1-piperidine-carboxylic acid are thus obtained and allowed to react without further purification

b) the compound obtained above is dissolved in 20 ml of dichloromethane and 0.6 g (6 mM) of triethylamine and 0.36 g (3.9 mM) of thioglycolic acid are then added. The mixture is agitated for 2 hours at ambient temperature and then washed with a dilute sodium hydroxide solution. The organic phase is dried over sodium sulphate and then concentrated under reduced pressure. The mixed residue is agitated with 25 ml of ethyl ether and the mixture is filtered. The solid is removed and 1 ml of a solution of hydrogen chloride in ethyl ether is added to the filtrate. The precipitate formed is filtered off and dried. 0.85 g of the product sought after is thus obtained in the form of a white powder (yield=67%).

M.Pt.=156° C.

Preparation XXXI ps 4-[[[2-[[2-[[(2,4-dichloro-3-methylphenyl)sulphonyl](2-phenylethyl)amino]acetyl]amino]acetyl][4-[[(1,1-dimethylethoxy)carbonyl]-amino]butyl]amino]methyl]-1-piperidinecarboxylic Acid, 1,1-dimethylethyl Ester

In performing analogously to preparation VI, starting with the compound obtained according to preparation XXX, the product sought after is obtained in the form of a yellow oil (yield=63%).

### EXAMPLE 25

N-[2-[(4-aminobutyl)(4-piperidinylmethyl)amino]-2-oxoethyl]-2-[[(2,4-dichloro-3-methylphenyl)sulphonyl](2-phenylethyl)amino]acetamide, Bis Trifluoroacetate

In performing analogously to Example 1, starting with the compound obtained according to preparation XXXXI, the product sought after is obtained in the form of a white solid (yield=58%).

M.Pt.=90° C.

Preparation XXXII

4-[[[4-(acetyloxy)butyl]amino]methyl]-1-piperidinecarboxylic Acid, 1,1-dimethylethyl Ester

a) a solution of 0.444 g (1 mM) of the compound obtained according to preparation XXIX is prepared in 5 ml of dimethylformamide. 0.48 g (2 mM) of 4-iodobutyl acetate and 0.69 g (5 mM) of potassium carbonate are added. The reaction mixture is agitated for 24 hours at ambient temperature and then diluted with 50 ml of ethyl acetate and washed successively with a 0.1N hydrochloric acid solution, a saturated sodium bicarbonate solution and then with water. The organic phase is dried over sodium sulphate and then concentrated under reduced pressure. The residue is purified by chromatography on silica gel in eluting with the aid of a cyclohexane/ethyl acetate mixture (6/4; v/v). 0.23 g of the 1,1-dimethylethyl ester of 4-[[[4-(acetyloxy)butyl][(2,4-dinitrophenyl)sulphonyl]amino]methyl]-1 piperidinecarboxylic acid are thus obtained in the form of an orange-yellow oil (yield=41%)

b) the compound obtained above is then treated with thioglycolic acid according to a method which is similar to the preparation XXX ((b), the purification being made on the non-salified compound, by means of a chromatography on silica gel in eluting with the aid of a dichloromethane/methanol/aqueous ammonia mixture (8/2/0.5; v/v/v).

The product is obtained in the form of a red oil (yield=91%).

NMR (¹H, 300 MHz, DMSO): 3.98 (t, 2H); 3.91 (m, 2H); 2.66 (m, 2H); 2.51 (m, 2H); 2.39 (d, 2H); 1.99 (s, 3H); 1.67-1.53 (m, 5H); 1.45 (m, 2H); 1.38 (s, 9H); 0.95 (m, 2H).

Preparation XXXIII

4-[[[4-(acetyloxy)butyl][2-[[2-[[(2,4-dichloro-3-methylphenyl)sulphonyl](2-phenylethyl)amino]acetyl]amino]acetyl]-amino]methyl]-1-piperidinecarboxylic acid, 1,1-dimethylethyl Ester

In performing analogously to preparation VI, starting with the preparation XXXII, the product sought after is obtained in the form of a badly-crystallised solid (yield=42%).

NMR (¹H, 300 MHz, DMSO): 8.18 (m, 1H); 7.86 (d, 1H); 7.57 (d, 1H); 7.12 (m, 3H); 7.04 (m, 2H); 4.17 (s, 2H); 3.99 (m, 2H); 3.93 (m, 4H); 3.45 (m, 2H); 3.24 (m, 2H); 3.13 (m, 2H); 2.74 (t, 2H); 2.60 (m, 2H); 2.35 (s, 3H); 1.96 (s, 3H); 1.76 (m, 1H); 1.55 (m, 4H); 1.48 (m, 2H); 1.35 (s, 9H); 0.98 (m, 2H).

### EXAMPLE 26

N-[2-[(4-hydroxybutyl)(4-piperidinylmethyl)amino]-2-oxoethyl]-2-[[(2,4-dichloro-3-methylphenyl)sulphonyl](2-phenylethyl)amino]acetamide, Trifluoroacetate

In performing analogously to Example 1, starting with the preparation XXXIII, the product sought after is obtained in the form of a white solid (yield=41%).

M.Pt.=80° C.

Preparation X)=IV

4-[[[4-(acetylamino)butyl]amino]methyl]-1-piperidine-carboxylic Acid, 1,1-dimethylethyl Ester

a) a solution of 0.64 g (3 mM) of the 1,1-dimethylethyl ester of 4-(aminomethyl)-1-piperidinecarboxylic acid is prepared in 10 ml of acetonitrile and 0.55 g (4 mM) of potassium carbonate and 0.7 g (2.5 mM) of N-(4-bromobutyl)phthalimide is added. The reaction mixture is heated under reflux for 16 hours and then concentrated under reduced pressure. The residue is taken up into solution in ethyl acetate and the organic phase is washed with a saturated sodium bicarbonate solution, and then dried over sodium sulphate and concentrated under reduced pressure. After purification by chromatography on silica gel in eluting with the aid of a dichloromethane/methanol/diisopropylamine mixture (90/10/2; v/v/v), N-[4-[[[1-[(1,1-dimethylethoxy)carbonyl]-4-piperidinyl]methyl]amino]butyl]phthalimide is obtained in the form of a badly-crystallised solid (yield=49%).

b) a solution of 0.49 g (1.18 mM) of the compound obtained above is prepared in 10 ml of tetrahydrofuran and 0.15 g (1.5 mM) of triethylamine and 0.24 g (1.4 mM) of benzyl chloroformate are added. The reaction mixture is maintained under agitation for 24 hours at ambient temperature, and then diluted with 60 ml of ethyl acetate. The organic phase obtained is washed with a dilute hydrochloric acid solution, and then with a saturated sodium bicarbonate solution, dried over sodium sulphate and concentrated under reduced pressure. The residue is purified by chromatography on silica gel in eluting with the aid of a cyclohexane/ethyl acetate mixture (6/4; v/v). 0.51 g of N-[4-[[[1-(1,1-dimethylethoxy)carbonyl]-4-piperidinyl]methyl]-[(phenylmethoxy)carbonyl]amino]butyl]phthalimide is thus obtained (yield=78%).

c) 0.11 g (0.2 mM) of the compound obtained above is dissolved in 1 ml of ethanol and 0.02 g (0.4 mM) of hydrazine hydrate is added. The mixture is heated for 3 hours under reflux and then concentrated under reduced pressure. The residue is purified by chromatography in eluting with the aid of a dichloromethane/methanol/diisopropylamine mixture (9/1/0; v/v/v). 66 mg of the 1,1-dimethylethyl ester of 4-[[(4-aminobutyl)[(phenylmethoxy)carbonyl]amino]methyl]-1-piperidinecarboxylic acid are thus obtained (yield=78%).

d) a solution of 0.17 g (0.4 mM) of the compound obtained according to the preceding step is prepared in 1 ml of pyridine and 51 mg (0.5 mM) of acetic anhydride are added. The mixture is agitated for 48 hours at ambient temperature and then diluted with 10 ml of ethyl acetate. The organic phase is washed in acid medium, and then with a sodium bicarbonate solution and dried over sodium sulphate. After concentration under reduced pressure, 0.18 g of the 1,1-dimethylethyl ester of 4-[[[4-(acetylamino)butyl][(phenylmethoxy)carbonyl]amino]methyl]-1-piperidine-carboxylic acid is obtained (yield=96%)

e) a solution of 1.04 g of the compound obtained according to the preceding step is prepared in 10 ml of ethanol and 100 mg of palladium on carbon (10% Pd) are added. The mixture is agitated under a hydrogen atmosphere at atmospheric pressure for 3 hours and the catalyst is then removed by filtration. The filtrate is concentrated under reduced pressure and 0.58 g of the compound sought after is thus obtained in the form of a pale yellow oil (yield=79%).

NMR ¹H (300 MHz, DMSO): 7.80 (m, 1H); 4.36 (m, 1H); 3.88 (m, 2H); 2.99 (m, 2H); 2.65 (m, 2H); 2.46 (m, 2H); 2.36 (d, 2H); 1.87 (s, 3H); 1.64 (m, 3H); 1.50 (m, 4H); 1.38 (s, 9H); 0.96 (m, 2H).

Preparation XXXV

4-[[[4-(acetylamino)butyl][2-[[2-[[(2,4-dichloro-3-methylphenyl)sulphonyl](2-phenylethyl)amino]acetyl]amino]acetyl]amino]methyl]-1-piperidinecarboxylic Acid, 1,1-dimethylethyl Ester

In performing analogously to preparation VI, starting with the compound obtained according to preparation XXXIV (e), the product sought after is obtained in the form of a pasty product (yield=87%).

NMR ¹H (250 MHz, DMSO): 8.16 (m, 1H); 7.86 (d, 2H); 7.82 (m, 1H); 7.57 (d, 2H); 7.12 (m, 3H); 7.04 (m, 2H); 4.17 (s, 2H); 3.95 (m, 4H); 3.46 (m, 4H); 3.23 (m, 2H); 3.15 (m, 2H); 3.04 (m, 2H); 2.73 (m, 2H); 2.38 (s, 3H); 1.78 (s, 3H); 1.52 (m, 5H); 1.40 (m, 2H); 1.37 (s, 9H); 1.02 (m, 2H).

### EXAMPLE 27

N-[2-[[4-(acetylamino)butyl](4-piperidinylmethyl)amino]-2-oxoethyl]-2-[[(2,4-dichloro-3-methylphenyl)sulphonyl](2-phenylethyl)amino]-acetamide, Trifluoroacetate

In performing analogously to Example 1, starting with the compound obtained according to preparation XXXV, the product sought after is obtained in the form of a white solid (yield=93%).

M.Pt.=100° C.

Preparation XXXVI

N-methyl-4-[[[3-(1-pyrrolidinyl)propyl]amino]methyl]benzamide

In performing analogously to preparation IV, starting with N-(3-aminopropyl)pyrrolidine and with 4-formyl-N-methyl-benzamide, the product sought after is obtained in the form of a yellow oil (yield=35%).

NMR ¹H (300 MHz, DMSO): 8.36 (m, 1H); 7.80 (d, 2H); 7.37 (d, 2H); 3.7 (s, 2H); 2.76 (d, 3H); 2.46 (m, 8H); 2.26 (m, 1H); 1.63 (m, 4H); 1.55 (m, 2H).

### EXAMPLE 28

4-[[[2-[[2-[[(2,4-dichloro-3-methylphenyl)sulphonyl](2-phenylethyl)-amin]acetyl]amin]acetyl][3-(1-pyrr lidinyl)prpyl]amin]methyl]-N-methyl-benzamide

In performing analogously to preparation VI, starting with the compound obtained according to preparation XXXVI, the product sought after is obtained in the form of a off-white solid (yield=31%).

M.Pt.=80° C.

### EXAMPLE 29

4-[[[2-[[2-[[(2,4-dichloro-3-methylphenyl)sulphonyl](2-phenylethyl)-amino]acetyl]amino]acetyl][3-(1-pyrrolidinyl)propyl]amino]methyl]-N-methyl-benzamide, Hydrochloride

In performing analogously to Example 6, starting with the compound obtained according to Example 28, the product sought after is obtained in the form of a fine white powder (yield=86%).

M.Pt.=110° C.

Preparation XXXVII

6-amino-N-[3-(1-pyrrolidinyl)propyl]nicotinamide

A solution of 0.8 g (6.24 mM) of 1-(3-aminopropyl)pyrrolidine is prepared in 40 ml of dichloromethane and 1.89 g (18.7 mM) of triethylamine and 1.2 g (6.24 mM) of 6-aminonicotinoyl chloride (in the form of hydrochloride) are added. The reaction mixture is agitated for 48 hours at ambient temperature. The precipitate formed is isolated by filtration, rinsed with dichloromethane and then dried. 0.75 g of the product sought after is obtained in the form of a beige solid (yield 50%).

M.Pt.=90° C.

Preparation XXXVIII

6-amino-N-[3-(1-pyrrolidinyl)propyl]-3-pyridinemethanamine

A suspension of 0.73 g (2.94 mM) of the compound obtained according to preparation XXXVII is prepared in 50 ml of dichloromethane. 10.3 ml (20.6 mM) of a 2M solution of borane/dimethylsulphide complex in tetrahydrofuran are added dropwise. The reaction mixture is agitated for 24 hours at ambient temperature. 15 ml of a 5N hydrochloric acid solution, and then 15 ml of water, and then 100 ml of methanol, are added. The mixture is agitated for 20 hours at ambient temperature and then concentrated under reduced pressure. The residue is purified by chromatography on NH₂ grafted silica (Lichroprep NH₂) in eluting with the aid of a dichloromethane/methanol mixture (98/2; v/v). 0.15 g of the product sought after are thus obtained in the form of a white solid (yield=22%).

M.Pt.=45-47° C.

### EXAMPLE 30

N-[2-[[(6-amino-3-pyridinyl)methyl][3-(1-pyrrolidinyl)propyl]amino]-2-oxoethyl]-2-[[(2,4-dichloro-3-methylphenyl)sulphonyl](2-phenylethyl)-amino]acetamide

In performing analogously to preparation VI, starting with the compound obtained according to preparation XXXVIII, the product sought after is obtained in the form of a white solid (yield=42%).

M.Pt.=80° C.

### EXAMPLE 31

N-[2-[[(6-amino-3-pyridinyl)methyl][3-(1-pyrrolidinyl)propyl]amino]-2-oxoethyl]-2-[[(2,4-dichloro-3-methylphenyl)sulphonyl](2-phenylethyl)-amino]acetamide, Dihydrochloride

In performing analogously to Example 6, starting with the compound obtained according to Example 30, the product sought after is obtained in the form of a white solid (yield=98%).

M.Pt.=142° C.

### EXAMPLE 32

N-[2-[bis[3-(dimethylamino)propyl]amino]-2-oxoethyl]-2-[[(2,4-dichloro-3-methylphenyl)sulphonyl](2-phenylethyl)amino]acetamide

In performing analogously to preparation VI, starting with bis[3-(dimethylamino)propyl]amine, the product sought after is obtained in the form of a yellow oil (yield=47%).

NMR ¹H (300 MHz, DMSO): 8.15 (m, 1H); 7.86 (d, 1H); 7.56 (d, 1H); 7.12 (m, 3H); 7.0 (m, 2H); 4.17 (s, 2H); 4.0 (d, 2H); 3.49 (t, 2H); 3.25 (m, 4H); 2.71 (t, 2H); 2.38 (s, 3H); 2.19 (m, 4H); 2.11 (s, 6H); 2.09 (s, 6H); 1.15 (m, 4H).

### EXAMPLE 33

N-[2-[bis[3-(dimethylamino)propyl]amino]-2-oxoethyl]-2-[[(2,4-dichloro-3-methylphenyl)sulphonyl](2-phenylethyl)amino]acetamide, Dihydrochloride

In performing analogously to Example 6, starting with the compound obtained according to Example 32, the product sought after is obtained in the form of a white, fine light solid (yield=90%).

M.Pt.=100° C.

Preparation XXXIX

4-[[[2-[[2-[[(2,4-dichloro-3-methylphenyl)sulphonyl](2-phenylethyl)amino]acetyl]amino]acetyl][3-(dimethylamino)propyl]amino]methyl]benzenecarboximidic Acid, Ethyl Ester, Hydrochloride.

A solution of 0.8 g (1.215 mM) of the compound obtained according to preparation XXIII is prepared in 50 ml of ethanol. This solution is cooled to 0° C. in an ice bath and then saturated with a flow of gaseous hydrogen chloride. The reaction mixture is then agitated for 48 hours at ambient temperature and then concentrated under reduced pressure. The precipitate formed is separated off by filtration, washed with ether and dried. 0.65 g of the product sought after is obtained in the form of white crystals (yield=68%).

M.Pt.=45-46° C.

### EXAMPLE 34

2-[[(2,4-dichloro-3-methylphenyl)sulphonyl](2-phenylethyl)amino]-N-[2-[[[4-(4,5-dihydro-1H-imidazol-2-yl)phenyl]methyl][3-(dimethylamino)-propyl]amino]-2-oxoethyl]acetamide

A solution of 0.057 g (0.95 mM) of ethylenediamine is prepared in 30 ml of ethanol. 0.64 g (0.91 mM) of the compound obtained according to preparation XXXIX in solution in 50 ml of ethanol is added, dropwise, at the temperature of reflux of ethanol. The reaction mixture is maintained under reflux for 48 hours and is then concentrated under reduced pressure. The residue is taken up in dichloromethane and the organic phase obtained is washed with water and then dried over sodium sulphate and concentrated under reduced pressure. The crude product obtained is purified by chromatography on NH₂ grafted silica in eluting with the aid of a toluene/2-propanol mixture (95/5; v/v). 0.18 g of the product sought after are thus obtained in the form of a white cream amorphous solid (yield=31%).

M.Pt.=75° C.

### EXAMPLE 35

2-[[(2,4-dichloro-3-methylphenyl)sulphonyl](2-phenylethyl)amino]-N-[2-[[[4-(4,5-dihydro-1H-imidazol-2-yl)phenyl]methyl][3-(dimethylamino)propyl]amino]-2-oxoethyl]acetamide, Dihydrochloride

In performing analogously to Example 6, starting with the compound obtained according to Example 34, the product sought after is obtained in the form of a fine white solid (yield=93%).

M.Pt.=142° C.

Preparation XL

N-[2-[[(4-cyanophenyl)methyl]methylamino]-2-oxoethyl]-2-[[(2,4-dichloro-3-methylphenyl)sulphonyl](2-phenylethyl)amino]acetamide

In performing analogously to preparation VI, starting with 4(methylaminomethyl)benzonitrile, the product sought after is obtained in the form of a white solid (yield=75%).

M.Pt.=72° C.

### EXAMPLE 36

N-[2-[[[4-[amino(hydroxyimino)methyl]phenyl]methyl]methylamino]-2-oxoethyl]-2-[[(2,4-dichloro-3-methylphenyl)sulphonyl](2-phenylethyl)amino]acetamide

In performing analogously to preparation VII, starting with the compound obtained according to preparation XL, the product sought after is obtained in the form of a white solid (yield=98%).

M.Pt.=97° C.

### EXAMPLE 37

N-[2-[[[4-[[(acet xy)imin](amin)methyl]phenyl]methyl]methylamin]-2-xoethyl]-2-[[(2,4-dichl ro-3-methylphenyl)sulph nyl](2-phenylethyl)amin]acetamide

In performing analogously to preparation VIII, starting with the compound obtained according to Example 36, the product sought after is obtained in the form of a white amorphous solid (yield=82%).

M.Pt.=50° C.

### EXAMPLE 38

N-[2-[[[4-(aminoiminomethyl)phenyl]methyl]methylamino]-2-oxoethyl]-2-[[(2,4-dichloro-3-methylphenyl)sulphonyl](2-phenylethyl)amino]-acetamide

In performing analogously to preparation IX, starting with the compound obtained according to Example 37, the product sought after is obtained in the form of a white amorphous solid (yield=95%).

M.Pt.=102° C.

### EXAMPLE 39

N-[2-[[[4-(aminoiminomethyl)phenyl]methyl]methylamino]-2-oxoethyl]-2-[[(2,4-dichloro-3-methylphenyl)sulphonyl](2-phenylethyl)amino]-acetamide, Hydrochloride

In performing analogously to Example 6, starting with the compound obtained according to Example 38, the product sought after is obtained in the form of a fine white amorphous solid (yield=88%).

M.Pt.=130° C.

Preparation XLI

4-[[[2-[[2-[[(2,4-dichloro-3-methylphenyl)sulphonyl](2-phenylethyl)amino]acetyl]amino]acetyl]methylamino]methyl]benzene-carboximidic Acid, Ethyl Ester, Hydrochloride.

In performing analogously to preparation XXXIX, starting with the compound obtained according to preparation XL, the product sought after is obtained in the form of a white solid (yield=60%).

M.Pt.=47-48° C.

### EXAMPLE 40

2-[[2,4-dichlor-3-methylphenyl)sulph nyl](2-phenylethyl)amin]-N-[2-[[[4-(4,5-dihydr-1H-imidazol-2-yl)phenyl]methyl]methylamin]-2-ox-ethyl]acetamide

In performing analogously to Example 34, starting with the compound obtained according to preparation XLI, the product sought after is obtained in the form of an ecru solid (yield=17%).

M.Pt.=80° C.

### EXAMPLE 41

2-[[2,4-dichloro-3-methylphenyl)sulphonyl](2-phenylethyl)amino]N-[2-[[[4-(4,5-dihydro-1H-imidazol-2-yl)phenyl]methyl]methylamino]-2-oxoethyl]acetamide, Hydrochloride

In performing analogously to Example 6, starting with the compound obtained according to Example 40, the product sought after is obtained in the form of a fine white solid (yield=90%).

M.Pt.=100° C.

Preparation XLII

N-[2-[[(4-cyanophenyl)methyl]amino]-2-oxoethyl]-2-[[(2,4-dichloro-3-methylphenyl)sulphonyl](2-phenylethyl)amino]acetamide

In performing analogously to preparation VI, starting with 4(aminomethyl)benzonitrile, the product sought after is obtained in the form of a white solid (yield=52%).

M.Pt.=82° C.

### EXAMPLE 42

N-[2-[[[4-[amino(hydroxyimino)methyl]phenyl]methyl]amino]-2-oxo-ethyl]-2-[[(2,4-dichloro-3-methylphenyl)sulphonyl](2-phenylethyl)amino]-acetamide

In performing analogously to preparation VII, starting with the compound obtained according to preparation XLII, the product sought after is obtained in the form of a white solid (yield=98%).

M.Pt.=100° C.

### EXAMPLE 43

N-[2-[[[4-[[(acetyl xy)imin](amin)methyl]phenyl]methyl]amino]-2-x ethyl]-2-[[(2,4-dichl r-3-methylphenyl)sulphonyl](2-phenylethyl)-amino]acetamide

In performing analogously to preparation VIII, starting with the compound obtained according to Example 42, the product sought after is obtained in the form of a white amorphous solid (yield=50%).

M.Pt.=104° C.

### EXAMPLE 44

N-2-[[[4-(aminoiminomethyl)phenyl]methyl]amino]-2-oxoethyl]-2-[[(2,4-dichloro-3-methylphenyl)sulphonyl](2-phenylethyl)-amino]acetamide

In performing analogously to preparation IX, starting with the compound obtained according to Example 43, the product sought after is obtained in the form of an off-white solid (yield=91%).

M.Pt.=130° C.

### EXAMPLE 45

N-2-[[[4-(aminoiminomethyl)phenyl]methyl]amino]-2-oxoethyl]-2-[[(2,4-dichloro-3-methylphenyl)sulphonyl](2-phenylethyl)amino]acetamide, Dihydrochloride

In performing analogously to Example 6, starting with the compound obtained according to Example 44, the product sought after is obtained in the form of a white solid (yield=83%).

M.Pt.=140° C.

Preparation XLIII

4-[[[(9H-fluoren-9-yl-methoxy)carbonyl]amino]methyl]-1-piperidine Carboxylic Acid, 1,1-dimethylethyl ester or [4-(Fmoc-aminomethyl)-1-Boc-piperidine]

A solution of 8.66 g (40.5 mM) of 4-(aminomethyl)-1-Boc-piperidine is prepared in 100 ml of DCM. 5.26 g (40.5 mM) of DIPEA and a solution of 10.47 g (40.5 mM) of 9H-fluoren-9-yl chloroformate (or Fmoc-Cl) in 50 ml of DCM are added. The reaction mixture is agitated at ambient temperature for 1 hour, washed with a saturated potassium bisulphate solution and then with water to neutrality. The organic phase is dried and then concentrated under reduced pressure. 16.9 g of the compound sought after are thus obtained which are used without other purification for the next step.

Preparation XLIV

(4-piperidinylmethyl)carbamic Acid, 9H-fluoren-9-yl-methyl Ester (or: 4-(Fmoc-aminomethyl)piperidine), Trifluoroacetate

A solution of 0.5 g (1.15 mM) of the compound obtained according to preparation XLIII is prepared in 10 ml of DCM and 3 ml of trifluoroacetic acid are added. The reaction mixture is agitated at ambient temperature for 2 hours and then concentrated under reduced pressure. The residue is taken up in toluene and once more concentrated under reduced pressure. The residue of evaporation is triturated in 10 ml of ethyl ether and the crystallised product formed is separated off by filtration, washed with 5 ml of ethyl ether and then dried under vacuum. 0.45 g of the product sought after are thus obtained in the form of a white solid (yield=87%).

M.Pt.=167° C.

Preparation XLV

[(1-Res-4-piperidinyl)methyl]carbamic Acid, 9H-fluoren-9-yl-methyl Ester (or: 4-(Fmoc-amino-methyl)1-Res-piperidine)

A suspension of 5.36 g of functionalised resin (styrene copolymer with 1% of divinylbenzene functionalised with a chlorotrityl group, loaded with 2.05 mM/g active chlorine obtained from the company Novabiochem) i.e. 11 mM, is prepared in 40 ml of DCM. 5.69 g (44 mM) of DIPEA, and then a solution of 7.43 g (16.5 mM) of the compound obtained according to preparation XLIV, are added. The reaction mixture is agitated with the aid of an orbital agitator for 18 hours at ambient temperature. The resin is separated off by filtration and rinsed successively with 10 ml of DMF, 10 ml of methanol, 10 ml of DCM, 10 ml of methanol, 10 ml of DCM and 10 ml of ethyl ether. After drying, the resin is used directly for carrying out the next step.

Preparation XLVI

2-[[[(1-Res-4-piperidinyl)methyl]amin]carb nyl]-1-piperidine carb xylic Acid, 1,1-dimethylethyl Ester or [N-[(1-Res-4-piperidinyl)-methyl]-1-B c-2-piperidinecarboxamide]

A suspension of 0.158 g (0.2 mM) of the resin obtained according to preparation XLV (grafting rate: 1.27 mM/g) is prepared in 5 ml of a 20% solution of piperidine in DMF. The reaction mixture is agitated for 5 hours at ambient temperature and then filtered. The resin is washed successively with 3 ml of DMF, 3 ml of DCM, and then 3 ml of DMF, and taken back up into suspension in 5 ml of DMF. 0.155 g (1.2 mM) of DIPEA, 0.138 g (0.6 mM) of N-Boc-2-piperidinecarboxylic acid, 0.076 g (0.6 mM) of HOBT and 0.075 g (0.6 mM) of DIC are then added. The mixture is agitated for 22 hours at ambient temperature and then filtered. The resin is washed successively with 3 ml of DMF, 3 ml of methanol, 3 ml of THF, 3 ml of methanol, 3 ml of THF and 5 ml of DCM, and then dried. The dried resin is used directly for the next step.

Preparation XLVII

2-[[[(1-Res-4-piperidinyl)methyl]amino]methyl]-1-piperidinecarboxylic Acid, 1,1-dimethylethyl Ester or 2-[[[(1-Res-4-piperidinyl)-methyl]amino]methyl]-1-Boc-piperidine

A suspension of 0.2 mM of the resin obtained according to preparation XLVI is prepared in 2 ml of THF and 0.083 g (0.8 mM) of trimethyl borate and then 2 ml of a 2M solution of borane/dimethylsulphide complex in ethyl ether, are added. The mixture is agitated at ambient temperature for 23 hours. The resin is separated off by filtration, washed with 3 ml of DCM, and then 3 ml of THF and allowed to react again in the presence of 2 ml of THF, 0.083 g (0.8 mM) of trimethyl borate and 2 ml of the 2M solution of borane/dimethylsulphide complex in ether, for 72 hours at ambient temperature. The resin is separated off by filtration, washed with 3 ml of DCM, and then with 3 ml of THF and agitated in the presence of 2 ml of THF and 0.47 g (8 mM) of propylamine for 24 hours. The resin is filtered off, washed with 3 ml of DMF, 3 ml of methanol, 3 ml of THF, 3 ml of methanol, 3 ml of THF and 4 ml of DCM. After drying, the resin is used directly in the next step.

Preparation XLVIII

2-[[[2-[[2-[[(2,4-dichl r-3-methylphenyl)sulph nyl](2-phenylethyl)amin]acetyl]amin]acetyl][(1-Res-4-piperidinyl)methyl]amino]-methyl]-1-piperidinecarboxylic Acid, 1,1-dimethylethyl Ester

A suspension of 0.2 mM of the resin obtained according to preparation XLVII is prepared in 4 ml of DMF. A solution of 0.081 g (0.6 mM) of HOBT in 1 ml of DMF, 0.076 g (0.6 mM) of DIC, 0.159 g (1.2 mM) of DIPEA and then a solution of 0.276 g of the acid obtained according to preparation III in 1 ml of DMF, are added. The reaction mixture is agitated for 12 hours at 50° C. and then 10 hours at ambient temperature. The resin is separated off by filtration and washed successively with 4 ml of DMF, 4 ml of DCM and then 4 ml of DMF. The resin is then subjected to a new cycle of coupling with the acid, under the same conditions, and then washed with 4 ml of DMF, 4 ml of methanol, 4 ml of THF, 4 ml of methanol, 4 ml of THF and 4 ml of DCM, and dried.

### EXAMPLE 46

2-[[(2,4-dichloro-3-methylphenyl)sulphonyl](2-phenylethyl)amino]-N-[2-oxo-2-[(2-piperidinylmethyl)(4-piperidinylmethyl)amino]ethyl]-acetamide, Bis Trifluoroacetate

A suspension of 0.2 mM of the resin obtained according to preparation XLVIII is prepared in 4 ml of DCM and 0.4 ml of trifluoroacetic acid are added. The mixture is agitated for 1.5 hours at ambient temperature and the resin is then filtered off and rinsed with the aid of 5 ml of DCM and then 5 ml of methanol. The combined filtrates are concentrated under a flow of nitrogen and the residue of evaporation is purified by preparative HPLC chromatography, with the aid of a 250×20 mm column packed with INERTSIL PREP ODS stationary phase obtained from the company G.L. Sciences Inc., and eluting with the aid of a water/acetonitrile mixture gradient and in the presence of 0.05% of trifluoroacetic acid. 117 mg the product sought after are thus obtained.

LC/MS (Grad. C): 2.32 minutes

In following the cycle of the steps of preparation XLVI to Example 46 and in modifying the nature of the acid employed in the preparation XLVI, the compounds of the following examples are obtained:

### EXAMPLE 47

2-[[(2,4-dichloro-3-methylphenyl)sulphonyl](2-phenylethyl)amino]-N-[2-oxo-2-[bis(4-piperidinylmethyl)amino]ethyl]acetamide, Bis Trifluoroacetate

LC/MS (Grad C): 2.17 minutes

### EXAMPLE 48

2-[[(2,4-dichloro-3-methylphenyl)sulphonyl](2-phenylethyl)amino]-N-[2-[[(1-methyl-4-piperidinyl)methyl](4-piperidinylmethyl)amino]-2-oxo-ethyl]acetamide, Bis Trifluoroacetate

LC/MS (Grad C): 2.20 minutes

### EXAMPLE 49

2-[[(2,4-dichloro-3-methylphenyl)sulphonyl](2-phenylethyl)amino]-N-[2-[[(1-ethyl-4-piperidine)methyl](4-piperidinylmethyl)amino]-2-oxoethyl]-acetamide, Bis Trifluoroacetate

LC/MS (Grad C): 2.30 minutes

Preparation IL

N-[(1-Res-4-piperidinyl)methyl]-2-nitro-benzenesulphonamide

A suspension of 0.158 g (0.2 mM) of the resin obtained according to preparation XLV (grafting rate is 1.27 mM/g is prepared in 5 ml of a 20% solution of piperidine in DMF. The reaction mixture is agitated at ambient temperature for 5 hours and then filtered. The resin is washed on a filter with 2 ml of DMF and then 2 ml of DCM, and then placed in suspension in 5 ml of DCM. 0.077 g (0.6 mM) of DIPEA and then a solution of 0.133 g (0.6 mM) of 2-nitro-benzenesulphonyl chloride in 2 ml of DCM are added. The reaction mixture is agitated for 30 hours at ambient temperature and then filtered. The resin is washed successively with each time 2 ml of DMF, methanol, THF, methanol, THF and DCM, and then used for the next step.

Preparation L

4-[2-[[(2-nitrophenyl)sulphonyl][1-Res-4-piperidinyl)methyl]amino]ethyl]-1-piperidinecarboxylic Acid, 1,1-dimethylethyl Ester

A suspension of 0.2 mM of the resin obtained according to preparation IL is prepared in 1 ml of THF and 0.52 g (2 mM) of triphenylphosphine in solution in 2 ml of THF are added, then a solution of 0.46 g (2 mM) of the 1,1-dimethylethyl ester of 4-(2-hydroxyethyl)-1-piperidinecarboxylic acid in 1 ml of THF, and then 0.20 g (1 mM) of DIAD. The mixture is agitated for 30 minutes at ambient temperature and 0.20 g (1 mM) of DIAD are added once more. The mixture is agitated at ambient temperature for 20 hours. The resin is filtered off, washed with 2 ml of DCM and then 2 ml of THF and subjected to a new alkylation cycle under the same conditions. The resin is then separated off and washed successively with each time 2 ml of DMF, methanol, THF, methanol, THF and DCM. The grafted resin thus obtained is used in the next step.

Preparation LI

4-[2-[[(1-Res-4-piperidinyl)methyl]amino]ethyl]-1-piperidinecarboxylic Acid, 1,1-dimethylethyl Ester

A suspension of 0.2 mM of the resin obtained according to preparation L is prepared in 5 ml of DMF. 0.22 g (2 mM) of thiophenol are added and then 0.12 g (1.2 mM) of triethylamine. The reaction mixture is agitated for 22 hours at ambient temperature, and the resin is then separated off by filtration and rinsed successively with each time 2 ml of DMF, methanol and THF. The resin is subjected a second time to the reaction cycle described above and it is washed on the filter successively with each time 2 ml of DMF, methanol, THF, methanol, THF and DCM. The resin thus obtained is used for the next step.

Preparation LII

4-[2-[[2-[[2-[[(2,4-dichloro-3-methylphenyl)sulphonyl](2-phenylethyl)amino]acetyl]amino]acetyl][(1-Res-4-piperidinyl)methyl]amino]-ethyl]-1-piperidinecarboxylic Acid, 1,1-dimethylethyl Ester

In performing analogously to preparation XLVIII, starting with the resin obtained according to preparation LI, the resin sought after is obtained.

### EXAMPLE 50

2-[[(2,4-dichloro-3-methylphenyl)sulphonyl](2-phenylethyl)amino]-N-[2-oxo-2-[[2-(4-piperidinyl)ethyl](4-piperidinylmethyl)amino]ethyl]-acetamide, Bis Trifluoroacetate

In performing analogously to Example 46, starting with the compound obtained according to preparation LII, the product sought after is obtained.

LC/MS (Grad B): 2.22 minutes

In performing analogously to the series of steps from preparation L to Example 50 and by modifying the nature of the amino-alcohol introduced in preparation L, the following compounds of the invention are obtained

### EXAMPLE 51

2-[[(2,4-dichloro-3-methylphenyl)sulphonyl](2-phenylethyl)amino]-N-[2-oxo-2-[[2-(1-piperidinyl)ethyl](4-piperidinylmethyl)amino]ethyl]-acetamide, Bis Trifluoroacetate

LC/MS (Grad B): 2.45 minutes

### EXAMPLE 52

2-[[(2,4-dichloro-3-methylphenyl)sulphonyl](2-phenylethyl)amino]-N-[2-oxo-2-[[2-(1-pyrrolinyl)ethyl](4-piperidinylmethyl)amino]ethyl]-acetamide, Bis Trifluoroacetate

LC/MS (Grad B): 2.42 minutes

### EXAMPLE 53

2-[[(2,4-dichloro-3-methylphenyl)sulphonyl](2-phenylethyl)amino]-N-[2-[[2-(hexahydro-1H-azepin-1-yl)ethyl](4-piperidinylmethyl)amino]-2-oxo-ethyl]acetamide, Bis Trifluoroacetate

LC/MS (Grad B): 2.57 minutes

### EXAMPLE 54

2-[[(2,4-dichloro-3-methylphenyl)sulphonyl](2-phenylethyl)amino]-N-[2-[[(1-methyl-3-piperidinyl)methyl](4-piperidinyl methyl)amin]-2-x-ethyl]acetamide, Bis Triflu r acetate

LC/MS (Grad B): 2.35 minutes

### EXAMPLE 55

2-[[(2,4-dichloro-3-methylphenyl)sulphonyl](2-phenylethyl)amino]-N-[2-[[(1-methyl-2-piperidinyl)methyl](4-piperidinyl methyl)amino]-2-oxoethyl]acetamide, Bis Trifluoroacetate

LC/MS (Grad B): 2.43 minutes

### EXAMPLE 56

2-[[(2,4-dichloro-3-methylphenyl)sulphonyl](2-phenylethyl)amino]-N-[2-[(3-aminopropyl)(4-piperidinylmethyl)amino]-2-oxoethyl]acetamide, bis trifluoroacetate

LC/MS (Grad B): 2.20 minutes

### EXAMPLE 57

2-[[(2,4-dichloro-3-methylphenyl)sulphonyl](2-phenylethyl)amino]-N-[2-[[3-(dimethylamino)propyl](4-piperidinylmethyl)amino]-2-oxoethyl]acetamide, Bis Trifluoroacetate

LC/MS (Grad A): 2.78 minutes

### EXAMPLE 58

2-[[(2,4-dichloro-3-methylphenyl)sulphonyl](2-phenylethyl)amino]-N-[2-[[2-[methyl(phenylmethyl)amino)ethyl](4-piperidinylmethyl)amino]-2-oxoethyl]acetamide, Bis Trifluoroacetate

LC/MS (Grad B): 2.78 minutes

### EXAMPLE 59

2-[[(2,4-dichloro-3-methylphenyl)sulphonyl](2-phenylethyl)amino]-N-[2-[[2-(4-methyl-1-piperazinyl)ethyl](4-piperidinylmethyl)amino]-2-oxo-ethyl]acetamide, Bis Trifluoroacetate

LC/MS (Grad B): 1.93 minutes

### EXAMPLE 60

2-[[(2,4-dichloro-3-methylphenyl)sulphonyl](2-phenylethyl)amino]-N-[2-oxo-2-[(4-piperidinylmethyl)(4-pyridinylmethyl)amin]ethyl]-acetamide, Bis Triflu r acetate

LC/MS (Grad A): 2.75 minutes

Preparation LIII

N-Res-1,4-butanediamine

A suspension of 7.32 g (15 mM) of functionalised resin (analogous to that employed for the preparation XLV) is prepared in 60 ml of DCM. 3.88 g (30 mM) of DIPEA and 2.65 g (30 mM) of 1,4-butanediamine are added. The reaction mixture is agitated for 18 hours at ambient temperature and the resin is then filtered off and washed successively with each time 15 ml of DCM, methanol, DCM and ethyl ether. The resin is then used for the next step.

Preparation LIV

N-Res-N′-[(2-nitrophenyl)sulphonyl]-1,4-butanediamine

A suspension of 0.2 mM of the resin obtained according to preparation LIII is prepared in 5 ml of DCM and 0.077 g (0.6 mM) of DIPEA are added, and then 0.133 g (0.6 mM) of 2-nitrobenzenesulphonyl chloride in 2 ml of DCM. The reaction mixture is agitated 30 hours at ambient temperature and then filtered. The resin is washed successively with each time 2 ml of DMF, methanol, THF, methanol, THF and DCM, and then used for the next step.

In then performing analogously to the steps described for the preparations L, LI, LII and Example 50, starting with the resin obtained according to preparation LIV and in appropriately modifying the nature of the alcohol in the first step, the following compounds of the invention are obtained:

### EXAMPLE 61

N-[2-[(4-aminobutyl)[2-(dimethylamino)ethyl]amino]-2-oxoethyl]-2-[[(2,4-dichloro-3-methylphenyl)sulphonyl](2-phenylethyl)amino]acetamide, Bis Trifluoroacetate

LC/MS (Grad A): 2.72 minutes

### EXAMPLE 62

N-[2-[(4-aminobutyl)[3-(dimethylamino)propyl]amino]-2-oxoethyl]-2-[[(2,4-dichl r-3-methylphenyl)sulph nyl](2-phenylethyl)amin]acetamide, Bis Triflu r acetate

LC/MS (Grad A): 2.70 minutes

### EXAMPLE 63

N-[2-[(4-aminobutyl)[2-(1-pyrrolidinyl)ethyl]amino]-2-oxoethyl]-2-[[(2,4-dichloro-3-methylphenyl)sulphonyl](2-phenylethyl)amino]acetamide, Bis Trifluoroacetate

LC/MS (Grad A): 2.80 minutes

### EXAMPLE 64

N-[2-[(4-aminobutyl)[2-(1-piperidinyl)ethyl]amino]-2-oxoethyl]-2-[[(2,4-dichloro-3-methylphenyl)sulphonyl](2-phenylethyl)amino]acetamide, Bis Trifluoroacetate

LC/MS (Grad A): 2.88 minutes

### EXAMPLE 65

N-[2-[(4-aminobutyl)[2-(4-piperidinyl)ethyl]amino]-2-oxoethyl]-2-[[(2,4-dichloro-3-methylphenyl)sulphonyl](2-phenylethyl)amino]acetamide, Bis Trifluoroacetate

LC/MS (Grad A): 2.72 minutes

### EXAMPLE 66

N-[2-[(4-aminobutyl)(3-piperidinylmethyl]amino]-2-oxoethyl]-2-[[(2,4-dichloro-3-methylphenyl)sulphonyl](2-phenylethyl)amino]acetamide, Bis Trifluoroacetate

LC/MS (Grad A): 2.72 minutes

### EXAMPLE 67

N-[2-[(4-aminobutyl)[(1-methyl-3-piperidinyl)methyl]amino]-2-oxo-ethyl]-2-[[(2,4-dichloro-3-methylphenyl)sulphonyl](2-phenylethyl)amino]acetamide, Bis Trifluoroacetate

LC/MS (Grad A): 2.75 minutes

### EXAMPLE 68

N-[2-[(4-aminobutyl)[(1-methyl-2-piperidinyl)methyl]amino]-2-x-ethyl]-2-[[(2,4-dichl r-3-methylphenyl)sulphonyl](2-phenylethyl)amin]acetamide, Bis Trifluor acetate

LC/MS (Grad A): 2.82 minutes

### EXAMPLE 69

N-[2-[(4-aminobutyl)[2-(hexahydro-1H-azepin-1-yl)ethyl]amino]-2-oxoethyl]-2-[[(2,4-dichloro-3-methylphenyl)sulphonyl](2-phenylethyl)amino]acetamide, Bis Trifluoroacetate

LC/MS (Grad A): 2.98 minutes

### EXAMPLE 70

N-[2-[(4-aminobutyl)[2-(4-methyl-1-piperazinyl)ethyl]amino]-2-oxo-ethyl]-2-[[(2,4-dichloro-3-methylphenyl)sulphonyl](2-phenylethyl)amino]acetamide, Bis Trifluoroacetate

LC/MS (Grad A): 2.48 minutes

### EXAMPLE 71

N-[2-[(4-aminobutyl)[2-(4-pyridinyl)ethyl]amino]-2-oxoethyl]-2-[[(2,4-dichloro-3-methylphenyl)sulphonyl](2-phenylethyl)amino]acetamide, Bis Trifluoroacetate

LC/MS (Grad C): 3.02 minutes

### EXAMPLE 72

N-[2-[(4-aminobutyl)[3-(4-pyridinyl)propyl]amino]-2-oxoethyl]-2-[[(2,4-dichloro-3-methylphenyl)sulphonyl](2-phenylethyl)amino]acetamide, Bis Trifluoroacetate

LC/MS (Grad A): 2.73 minutes

### EXAMPLE 73

N-[2-[(4-aminobutyl)[3-(3-pyridinyl)propyl]amino]-2-oxoethyl]-2-[[(2,4-dichloro-3-methylphenyl)sulphonyl](2-phenylethyl)amino]acetamide, Bis Trifluoroacetate

LC/MS (Grad A): 2.75 minutes cl EXAMPLE 74

N-[2-[(4-aminobutyl)[3-(2-pyridinyl)propyl]amino]-2-oxoethyl]-2-[[(2,4-dichior-3-methylphenyl)sulph nyl](2-phenylethyl)amin]acetamide, Bis Trifluor acetate

LC/MS (Grad A): 2.77 minutes

### EXAMPLE 75

N-[2-[(4-aminobutyl)[2-[[(ethylamino)carbonyl]oxy]ethyl]amino]-2-oxoethyl]-2-[[(2,4-dichloro-3-methylphenyl)sulphonyl](2-phenyl-ethyl)amino]acetamide, Trifluoroacetate

LC/MS (Grad D): 5.93 minutes

### EXAMPLE 76

N-[2-[(4-aminobutyl)[3-[[(ethylamino)carbonyl]oxy]propyl]amino]-2-oxoethyl]-2-[[(2,4-dichloro-3-methylphenyl)sulphonyl]-(2-phenylethyl)-amino]acetamide, Trifluoroacetate

LC/MS (Grad D): 5.97 minutes

### EXAMPLE 77

N-[2-[(4-aminobutyl)[4-[[(ethylamino)carbonyl]oxy]butyl]amino]-2-oxoethyl]-2-[[(2,4-dichloro-3-methylphenyl)sulphonyl](2-phenylethyl)-amino]acetamide, Trifluoroacetate

LC/MS (Grad D): 6.12 minutes

### EXAMPLE 78

N-[2-[(4-aminobutyl)[3-[[(methoxy)carbonyl]amino]propyl]amino]-2-oxoethyl]-2-[[(2,4-dichloro-3-methylphenyl)sulphonyl]-(2-phenylethyl)-amino]acetamide, Trifluoroacetate

LC/MS (Grad D): 5.82 minutes

### EXAMPLE 79

N-[2-[(4-aminobutyl)[4-[[(methoxy)carbonyl]amino]butyl]amino]-2-oxoethyl]-2-[[(2,4-dichloro-3-methylphenyl)sulphonyl](2-phenylethyl)-amino]acetamide, Trifluoroacetate

LC/MS (Grad D): 5.82 minutes

### EXAMPLE 80

### N-[2-[(4-aminobutyl)(3-aminopropyl)amino]-2-oxoethyl]-2-[[(2,4-dichlor-3-methylphenyl)sulph nyl](2-phenylethyl)amino]acetamide, Bis Triflu r acetate

LC/MS (Grad A): 2.65 minutes

Preparation LV

5-[3-[[4-(1-pyrrolidinyl)butyl]amino]propyl]-1-(triphenylmethyl)-1-H-imidazole

In performing analogously to preparation XIV, starting with 1-(4-aminobutyl)pyrrolidine and 1-(triphenylmethyl)-1-H-imidazole-5-propanal, the product sought after is obtained in the form of a yellow oil (yield 35%).

n_{D}²²=1.596

Preparation LVI

2-[[(2,4-dichloro-3-methylphenyl)sulphonyl](2-phenylethyl)amino]-N-[2-oxo-2-[[4-(1-pyrrolidinyl)butyl][3-[1-(triphenylmethyl)-1H-imidazol-5-yl]propyl]amino]ethyl]acetamide.

A solution of 1.84 g (4 mM) of the acid obtained according to preparation III is prepared in 20 ml of acetonitrile and a solution of 1.97 g (4 mM) of the amine obtained in preparation LV in 20 ml of acetonitrile is added, and then 1.67 g (4.4 mM) of BTUH (O-benzotriazol-1-yl-N,N,N′,N′-tetramethyluronium hexafluorophosphate) and then 0.59 g (4.4 mM) of HOBT and 0.57 g (4.4 mM) of diisopropylethylamine. The reaction mixture is agitated for 20 hours at ambient temperature, and then concentrated under reduced pressure. The residue is taken up in dichloromethane and the organic phase obtained is washed with the aid of a dilute sodium hydroxide solution, and then with water, and then dried over sodium sulphate and concentrated under reduced pressure. The residue is purified by chromatography on silica gel in eluting with the aid of a dichloromethane/methanol mixture (95/5; v/v). 3.6 g of the product sought after are thus obtained in the form of an amorphous solid (yield=87%).

M.Pt.=72° C.

### EXAMPLE 81

2-[[(2,4-dichloro-3-methylphenyl)sulphonyl](2-phenylethyl)amino]-N-[2-[[3-(1H-imidazol-5-yl)propyl][4-(1-pyrrolidinyl)butyl]amino]-2-oxo-ethyl]acetamide

A solution of 3.6 g (3.86 mM) of the product obtained according to preparation LV is prepared in 30 ml of dichloromethane and 0.42 g (3.86 mM) of anisole are added and then 15 ml of trifluoroacetic acid. The reaction mixture is agitated for 20 hours at ambient temperature and then concentrated under reduced pressure. 100 ml of toluene are added to the residue and concentration is carried out once more under reduced pressure so as to drive off the trifluoroacetic acid. The residue is purified by chromatography on silica gel in eluting with the aid of a dichloromethane/methanol/aqueous ammonia mixture (90/10/1; v/v/v). 2.1 g of the product sought after are thus obtained in the form of a white amorphous solid (yield=78%).

M.Pt.=114° C.

### EXAMPLE 82

2-[[(2,4-dichloro-3-methylphenyl)sulphonyl](2-phenylethyl)amino]-N-[2-[[3-(1H-imidazol-5-yl)propyl][4-(1-pyrrolidinyl)butyl]amino]-2-oxo-ethyl]acetamide, Dihydrochloride

In performing analogously to Example 6, starting with the compound obtained according to Example 81, the product sought after is obtained in the form of a white solid (yield=98%).

M.Pt.=122° C.

Preparation LVII

2,4-dichloro-N,3-dimethylbenzenesulphonamide

In performing analogously to preparation I, starting with methylamine hydrochloride and an excess of triethylamine, the product sought after is obtained in the form of a white solid (yield=83%)

M.Pt.=112° C.

Preparation LVIII

N-[2-[[(2,4-dichloro-3-methylphenyl)sulphonyl]methylamino]acetyl]-glycine, Ethyl Ester

In performing analogously to preparation II, starting with the compound obtained according to preparation LVII, the product sought after is obtained in the form of a white solid (yield=51%)

M.Pt.=120° C.

Preparation LIX

N-[2-[[(2,4-dichloro-3-methylphenyl)sulphonyl]methylamino]acetyl]-glycine

A solution of 4.65 g (11 mM) of the ester obtained according to preparation LVIII is prepared in 100 ml of THF and a solution of 0.96 g (23 mM) of lithium hydroxide in 20 ml of water is added. The reaction mixture is agitated for 3 hours at 50° C. and then concentrated under reduced pressure. The residue is taken up with water and acidified at 5° C. with a 1N hydrochloric acid solution. The mixture is extracted with DCM and the organic phase obtained is washed with water, dried and concentrated under reduced pressure. 3.79 g of the product sought after are thus obtained in the form of a white solid (yield=93%).

M.Pt.=154° C.

Preparation LX

[(4-cyanophenyl)methyl]methylcarbamic Acid, Phenylmethyl Ester

A mixture of 7 g (47.9 mM) of [(4-cyanophenyl)methyl]methanamine is prepared in 60 ml of DCM and 5.8 g (57.5 mM) of triethylamine are added. The mixture is cooled to 0° C. and a solution of 9.8 g (57.5 mM) of benzyl chloroformate in 20 ml of DCM is added dropwise. The mixture is then agitated for 20 hours at ambient temperature and then washed with a 0.1 N hydrochloric acid solution, and then with water, dried over sodium sulphate and concentrated under reduced pressure. The residue is purified by chromatography on silica gel in eluting with the aid of a toluene/ethyl acetate mixture (95/5; v/v). 11.4 g of the product sought after are thus obtained in the form of an oil (yield=87%).

n_{D}²²=1.564

Preparation LXI

[[4-(4,5-dihydro-1H-imidazol-2-yl)phenyl]methyl]methylcarbamic Acid, Phenylmethyl Ester

A mixture of 11.3 g (40 mM) of the compound obtained according to preparation LX is prepared in 40 ml of ethylenediamine and 0.64 g (20 mM) of flowers of sulphur are added. The reaction mixture is agitated for 2 hours at 100° C. and then cooled. Water is added and extraction is carried out with ethyl acetate. The organic phase is washed with water, dried over sodium sulphate and concentrated under reduced pressure. The residue is purified by chromatography on silica gel in eluting with the aid of a dichloromethane/methanol/aqueous ammonia mixture (95/5/0.05; v/v/v). 11 g of product sought after are thus obtained in the form of a white solid (yield=85%).

M.Pt.=84° C.

Preparation LXII

4,5-dihydro-2-[4-[[methyl[(phenylmethoxy)carbonyl]amino]methyl]phenyl]-1H-imidazole-1-carboxylic acid, 1,1-dimethylethyl Ester

A solution of 3.22 g (10 mM) of the compound obtained according to preparation LXI is prepared in 45 ml of DCM, and 1.34 g (11 mM) of N,N-dimethylaminopyridine are added, a solution of 2.4 g (11 mM) of di-tert-butyl dicarbonate in 45 ml of DCM is then added dropwise. The reaction mixture is agitated for 2 hours at ambient temperature and then washed with the aid of a 0.5 N hydrochloric acid solution, and then with water. The organic phase is dried over sodium sulphate and then concentrated under reduced pressure.

The residue is crystallised in isopropyl ether and then filtered off and dried. 4 g of the product sought after are thus obtained in the form of fine white crystals (yield=94%).

M.Pt.=124° C.

Preparation LXIII

4,5-dihydro-2-[4-[(methylamino)methyl]phenyl]-1H-imidazole-1-carboxylic Acid, 1,1-dimethylethyl Ester

A mixture of 4.23 g (10 mM) of the compound obtained according to preparation LXII is prepared in 80 ml of methanol and 0.4 g of palladium on carbon (10% Pd) is added. The mixture is agitated under a hydrogen atmosphere at ambient temperature and at atmospheric pressure for 2 hours. The catalyst is removed by filtration and the filtrate is then concentrated under reduced pressure. The residue is purified by chromatography on silica gel in eluting with the aid of a dichloromethane/methanol/aqueous ammonia mixture (90/10/0.1; v/v/v). 2.5 g of the product sought after are thus obtained in the form of an off-white solid (yield=90%).

M.Pt.=65° C.

Preparation LXIV

2-[4-[[[2-[[2-[[(2,4-dichloro-3-methylphenyl) sulphonyl]methylamino]acetyl]amino]acetyl]methylamino]methyl]phenyl]-4,5-dihydro-1H-imidazole-1-carboxylic Acid, 1,1-dimethylethyl Ester

In performing analogously to preparation VI, starting with the compounds obtained according to preparations LIX and LXIII, the product sought after is obtained in the form of a white solid (yield=90%).

M.Pt.=61° C.

### EXAMPLE 83

2-[[(2,4-dichloro-3-methylphenyl)sulphonyl]methylamino]-N-[2-[[[4-(4,5-dihydro-1H-imidazol-2-yl)phenyl]methyl]methylamino]-2-oxoethyl]acetamide

In performing analogously to Example 1, starting with the compound obtained according to preparation LXIV, the product sought after is obtained after purification by chromatography on silica gel (eluent: dichloromethane/methanol/aqueous ammonia; 95/5/0.1; v/v/v), in the form of a white solid (yield=98%).

M.Pt.=72° C.

### EXAMPLE 84

2-[[(2,4-dichloro-3-methylphenyl)sulphonyl]methylamino]-N-[2-[[[4-(4,5-dihydro-1H-imidazol-2-yl)phenyl]methyl]methylamino]-2-oxoethyl]-acetamide, Hydrochloride

In performing analogously to Example 6, starting with the compound obtained according to Example 83, the product sought after is obtained in the form of a white powder (yield=88%).

M.Pt.=162° C.

Preparation LXV

2,4-dichl r-3-methyl-N-(1,1-dimethylethyl)benzene-sulphonamide

In performing analogously to preparation I, starting with tert-butylamine, the product sought after is obtained in the form of a white solid (yield=84%).

M.Pt.=150° C.

Preparation LXVI

N-[2-[[(2,4-dichloro-3-methylphenyl)sulphonyl][1,1-dimethylethyl]amino]acetyl]glycine, Ethyl Ester

A solution of 3 g (10 mM) of the compound obtained according to preparation LXV is prepared in 80 ml of anhydrous DMF and 0.264 g (11 mM) of sodium hydride is added. The mixture is agitated for 1 hour at ambient temperature and a solution of 2.98 g (11 mM) of the ethyl ester of N-(2-iodoacetyl)glycine is then added dropwise. The reaction mixture is agitated for 15 hours at ambient temperature and then poured onto water and extracted with ethyl acetate. The organic phase is washed with water, dried and concentrated under reduced pressure. The residue is purified by chromatography on silica gel in eluting with the aid of a methylcyclohexane/ethyl acetate mixture (6/4; v/v). 1.87 g of the product sought after are thus obtained in the form of a white solid (yield=42%).

M.Pt.=180° C.

Preparation LXVII

N-[2-[[(2,4-dichloro-3-methylphenyl)sulphonyl][1,1-dimethylethyl]amino]acetyl]glycine

In performing analogously to preparation LIX, starting with the compound obtained according to preparation LXVI, the product sought after is obtained in the form of a white powder (yield=80%).

M.Pt.=178° C.

Preparation LXVIII

2-[[(2,4-dichloro-3-methylphenyl)sulphonyl][1,1-dimethylethyl]amino]-N-[2-oxo-2-[[4-(1-pyrrolidinyl)butyl][3-[1-(triphenylmethyl)-1H-imidazol-5-yl]propyl]amino]ethyl]acetamide

In performing analogously to preparation LVI, starting with the acid obtained according to preparation LXVII, the product sought after is obtained in the form of a beige solid (yield=95%).

M.Pt.=85° C.

### EXAMPLE 85

2-[[(2,4-dichloro-3-methylphenyl)sulphonyl]amino]-N-[2-[[3-(1H-imidazol-5-yl)propyl][4-(1-pyrrolidinyl)butyl]amino]-2-oxoethyl]acetamide

In performing analogously to Example 81, starting with the compound obtained according to preparation LXVIII, the product sought after is obtained in the form of a white amorphous solid (yield=67%).

M.Pt.=88° C.

### EXAMPLE 86

2-[[(2,4-dichloro-3-methylphenyl)sulphonyl]amino]-N-[2-[[3-(1H-imidazol-5-yl)propyl][4-(1-pyrrolidinyl)butyl]amino]-2-oxoethyl]acetamide, Dihydrochloride

In performing analogously to Example 6, starting with the compound obtained according to Example 85, the product sought after is obtained in the form of an off-white powder (yield=75%).

M.Pt.=55° C.

Preparation LXIX

2,4-dichloro-N-(2-methoxymethyl)-3-methyl-benzenesulphonamide

In performing analogously to preparation I, starting with 2-methoxyethylamine, the product sought after is obtained in the form of a yellow oil (yield=78%).

¹H NMR (300 MHz, DMSO) δ: 8.00 (s, 1H, NH); 7.83 (d, 1H); 7.63 (d, 1H); 3.27 (t, 2H); 3.07 (s, 3H); 3.02 (t, 2H); 2.49 (s, 3H).

Preparation LXX

N-[2-[[(2,4-dichloro-3-methylphenyl)sulphonyl](2-methoxyethyl)amino]acetyl]glycine, Ethyl Ester

In performing analogously to preparation II, starting with the compound obtained according to preparation LXIX, the product sought after is obtained in the form of a white solid (yield=87%).

M.Pt.=108° C.

Preparation LXXI

N-[2-[[(2,4-dichloro-3-methylphenyl)sulphonyl](2-methoxyethyl)amino]acetyl]glycine In performing analogously to preparation LIX, starting with the compound obtained according to preparation LXX, the product sought after is obtained in the form of a white solid (yield=86%).

M.Pt.=140° C.

Preparation LXXII

2-[[(2,4-dichloro-3-methylphenyl)sulphonyl](2-methoxyethyl)amino]-N-[2-oxo-2-[[4-(1-pyrrolidinyl)butyl][3-[1-(triphenylmethyl)-1H-imidazol-5-yl]propyl]amino]ethyl]acetamide.

In performing analogously to preparation LVI, starting with the acid obtained according to preparation LXXI, the product sought after is obtained in the form of a fine yellow solid (yield=66%).

M.Pt.=50° C.

### EXAMPLE 87

2-[[(2,4-dichloro-3-methylphenyl)sulphonyl](2-methoxyethyl)amino]-N-[2-[[3-(1H-imidazol-5-yl)propyl][4-(1-pyrrolidinyl)butyl]amino]-2-oxo-ethyl]acetamide

In performing analogously to Example 81, starting with the compound obtained according to preparation LXXII, the product sought after is obtained in the form of a white ecru solid (yield=70%).

M.Pt.=50° C.

### EXAMPLE 88

2-[[(2,4-dichloro-3-methylphenyl)sulphonyl](2-methoxyethyl)amino]-N-[2-[[3-(1H-imidazol-5-yl)propyl][4-(1-pyrrolidinyl)butyl]amino]-2-oxo-ethyl]acetamide, Dihydrochloride

In performing analogously to Example 6, starting with the compound obtained according to Example 87, the product sought after is obtained in the form of a white solid (yield=97%).

M.Pt.=92° C.

Preparation LXXIII

2,4-dichloro-3-methyl-N-[2-(3-pyridinyl)ethyl)benzene-sulphonamide

In performing analogously to preparation I, starting with 2-(3-pyridinyl)ethylamine, the product sought after is obtained in the form of a white solid (yield=80%).

M.Pt.=106° C.

Preparation LXXIV

N-2-[2-[[(2,4-dichloro-3-methylphenyl)sulphonyl][2-(3-pyridinyl)ethyl]-amino]acetyl]glycine, 1,1-dimethylethyl Ester

In performing analogously to preparation II, starting with the compound obtained according to preparation LXXIII, the product sought after is obtained in the form of a white ecru solid (yield=36%).

M.Pt.=130° C.

Preparation LXXV

N-2-[2-[[(2,4-dichloro-3-methylphenyl)sulphonyl][2-(3-pyridinyl)ethyl]-amino]acetyl]glycine, Hydrochloride

A solution of 0.53 g (1.02 mM) of the ester obtained according to preparation LXXIV is prepared in 10 ml of dichloromethane and 10 ml of a saturated solution of hydrogen chloride in dioxane is added. The reaction mixture is agitated for 10 days at ambient temperature and the precipitate formed is then filtered off and rinsed with dichloromethane and dried. 0.43 g of the product sought after is thus obtained in the form of a white solid (yield=84%).

M.Pt.=154° C.

Preparation LXXVI

2-[[(2,4-dichloro-3-methylphenyl)sulphonyl][2-(3-pyridinyl)ethyl]amino]-N-[2-oxo-2-[[4-(1-pyrrolidinyl)butyl][3-[1-(triphenylmethyl)-1H-imidazol-5-yl]propyl]amino]ethyl]acetamide.

In performing analogously to preparation LVI, starting with the compound obtained according to preparation LXXV, the product sought after is obtained in the form of a white solid (yield=63%).

M.Pt.=82° C.

### EXAMPLE 89

2-[[(2,4-dichloro-3-methylphenyl)sulphonyl][2-(3-pyridinyl)ethyl]amino]-N-[2-[[3-(1H-imidazol-5-yl)propyl][4-(1-pyrrolidinyl)butyl]amino]-2-oxoethyl]acetamide

In performing analogously to Example 81, starting with the compound obtained according to preparation LXXVI, the product sought after is obtained in the form of a beige paste (yield=99%).

¹H NMR (300 MHz, DMSO) δ: 8.28 (m, 2H); 8.21 (m, 1H); 7.84 (d, 1H) 7.56 (d, 2H); 7.07 (t, 1H); 6.81 (d, 1H); 4.21 (d, 2H); 3.98 (dd, 2H); 0.49 (t, 2H); 3.27 (d, 4H); 3.15 (s, 4H); 3.03 (2H); 2.74 (t, 2H); 2.49 (m 2H); 2.34 (s, 3H); 1.88 (m, 6H); 1.53 (m, 4H).

### EXAMPLE 90

2-[[(2,4-dichloro-3-methylphenyl)sulphonyl][2-(3-pyridinyl)ethyl]amino]-N-[2-[[3-(1H-imidazol-5-yl)propyl][4-(1-pyrrolidinyl)butyl]amino]-2-oxoethyl]acetamide, Tris Trifluoroacetate

A solution of 0.14 g (0.202 mM) of the compound obtained according to Example 89 is prepared in 2 ml of methanol and 50 μl of trifluoroacetic acid are added. The reaction mixture is agitated for 15 min at ambient temperature and then concentrated under reduced pressure. The residue is taken up in 20 ml of water and the solution obtained is lyophilised. 0.17 g of the product sought after is thus obtained in the form of a white solid (yield=81%).

M.Pt.=60° C.

Preparation LXXVII

2-[[(2,4-dichloro-3-methylphenyl)sulphonyl]amino]acetamide

In performing analogously to preparation I, starting with 2-aminoacetamide, the product sought after is obtained in the form of a white solid (yield=60%).

M.Pt.=180° C.

Preparation LXXVIII

N-[2-[(2-amino-2-oxoethyl)[(2,4-dichloro-3-methylphenyl)sulphonyl]-amino]acetyl]glycine, Ethyl Ester

In performing analogously to preparation II, starting with the compound obtained according to preparation LXXVII, the product sought after is obtained in the form of a white powder (yield=76%).

M.Pt.=190° C.

Preparation LXXIX

N-[2-[(2-amino-2-oxoethyl)[(2,4-dichloro-3-methylphenyl)sulphonyl]amino]acetyl]glycine

In performing analogously to preparation LIX, starting with the preparation LXXVIII, the product sought after is obtained in the form of a white solid (yield=43%).

M.Pt.=94° C.

Preparation LXXX

2-[4-[[[2-[[2-[(2-amino-2-oxoethyl)[(2,4-dichloro-3-methylphenyl)sulphonyl]amino]acetyl]amino]acetyl]methylamino]methyl]phenyl]-4,5-dihydro-1H-imidazole-1-carboxylic Acid, 1,1-dimethylethyl Ester

In performing analogously to preparation VI, starting with the compounds obtained according to preparations LXXIX and LXIII, the product sought after is obtained in the form of a white solid (yield=53%).

M.Pt.=118° C.

### EXAMPLE 91

2-[(2-amino-2-oxoethyl)[(2,4-dichloro-3-methylphenyl)sulphonyl]amino]-N-[2-[[[4-(4,5-dihydro-1H-imidazol-2-yl)phenyl]methyl]methylamino]-2-oxoethyl]acetamide, Trifluoroacetate

In performing analogously to Example 1, starting with the compound obtained according to preparation LXXX, the product sought after is obtained in the form of a white solid (yield=83%).

M.Pt.=130° C.

Preparation LXXI

N-methyl-2,3,4-trichlorobenzenesulphonamide

In performing analogously to preparation LVII, starting with 2,3,4-trichlorobenzenesulphonyl chloride, the product sought after is obtained in the form of a beige solid (yield=53%).

M.Pt.=134° C.

Preparation LXXXII

N-[2-[methyl-[(2,3,4-trichlorophenyl)sulphonyl]amino]acetyl]glycine, Ethyl Ester

In performing analogously to preparation II, starting with the compound obtained according to preparation LXXXI and ethyl N-(iodoacetyl)glycinate, the product sought after is obtained in the form of a white solid (yield=80%).

M.Pt.=140° C.

Preparation LXXXIII

N-[2-[methyl-[(2,3,4-trichlorophenyl)sulphonyl]amino]acetyl]glycine

In performing analogously to preparation LIX, starting with the compound obtained according to preparation LXXXII, the product sought after is obtained in the form of a white powder (yield=93%).

M.Pt.=132° C.

Preparation LXXXIV 2-[methyl[(2,3,4-trichlorophenyl)sulphonyl]amino]-N-[2-oxo-2-[[4-(1-pyrrolidinyl)butyl][3-[1-(triphenylmethyl)-1H-imidazol-5-yl]propyl]-amino]ethyl]acetamide

In performing analogously to preparation LVI, starting with the acid obtained according to preparation LXXXIII, the product sought after is obtained in the form of a beige solid (yield=68%).

M.Pt.=120° C.

### EXAMPLE 92

N-[2-[[3-(1H-imidazol-5-yl)propyl][4-(1-pyrrolidinyl)butyl]amino]-2-oxoethyl]-2-[methyl[(2,3,4-trichlorophenyl)sulphonyl]amino]acetamide, Bis Trifluoroacetate

In performing analogously to Example 81, but in purifying the crude compound with the aid of a dichloromethane/methanol mixture (90/10; v/v), the product sought after is obtained in the form of a beige solid (yield=71%).

M.Pt.=76° C.

Preparation LXXXV

N-(2-propenyl)-2,4-dichloro-3-methylbenzenesulphonamide

In performing analogously to preparation I, starting with allylamine, the product sought after is obtained in the form of a white solid (yield=77%).

M.Pt.=91° C.

Preparation LXXXVI

N-[2-[[(2,4-dichloro-3-methylphenyl)sulphonyl]2-propenylamino]acetyl]-glycine, Ethyl Ester

In performing analogously to preparation II, starting with the compound obtained according to preparation LXXXV, the product sought after is obtained in the form of a white solid (yield=87%).

M.Pt.=81° C.

Preparation LXXXVII

N-[2-[[(2,4-dichloro-3-methylphenyl)sulphonyl]2-propenylamino]acetyl]glycine,

In performing analogously to preparation LIX, starting with the compound obtained according to preparation LXXXV, the product sought after is obtained in the form of a white solid (yield=99%).

M.Pt.=138° C.

Preparation LXXXVIII

2-[[(2,4-dichloro-3-methylphenyl)sulphonyl]2-propenylamino]-N-[2-oxo-2-[[4-(1-pyrrolidinyl)butyl][3-[1-(triphenylmethyl)-1H-imidazol-5-yl]-propyl]amino]ethyl]acetamide

In performing analogously to preparation LVI, starting with the compound obtained according to preparation LXXXVII, the product sought after is obtained in the form of a white solid (yield=40%).

M.Pt.=60° C.

### EXAMPLE 93

2-[[(2,4-dichloro-3-methylphenyl)sulphonyl]2-propenylamino]-N-[2-[[3-(1H-imidazol-5-yl)propyl][4-(1-pyrrolidinyl)butyl]amino]-2-oxoethyl]acetamide

In performing analogously to Example 81, starting with the compound obtained according to preparation LXXXVIII, the product sought after is obtained in the form of an amorphous solid (yield=75%).

M.Pt.=50° C.

### EXAMPLE 94

2-[[(2,4-dichloro-3-methylphenyl)sulphonyl]2-propenylamino]-N-[2-[[3-(1H-imidazol-5-yl)propyl][4-(1-pyrrolidinyl)butyl]amino]-2-oxoethyl]-acetamide, Dihydrochloride

In performing analogously to Example 6, starting with the compound obtained according to Example 93, the product sought after is obtained in the form of a white solid (yield=80%).

M.Pt.=90° C.

Preparation LXXXIX

N-methyl-2,6-dichlorobenzenesulphonamide

In performing analogously to preparation LVII, starting with 2,6-benzenesulphonyl chloride, the product sought after is obtained in the form of a white ecru solid (yield=99%).

M.Pt.=115° C.

Preparation XC

N-[2-[[(2,6-dichlorophenyl)sulphonyl]methylamino]acetyl]glycine, Ethyl Ester

In performing analogously to preparation II, starting with the compound obtained according to preparation LXXXIX, the product sought after is obtained, which is used without further purification for the next operation.

Preparation XCI

N-[2-[[(2,6-dichlorophenyl)sulphonyl]methylamino]acetyl]glycine

In performing analogously to preparation LIX, starting with the ester obtained according to preparation XC, the product sought after is obtained in the form of a white ecru solid (yield=76%).

M.Pt.=157° C.

Preparation XCII

2-[4-[[[2-[[2-[[(2,6-dichlorophenyl)sulphonyl]methylamino]acetyl]-amino]acetyl]methylamino]methyl]phenyl]-4,5-dihydro-1H-imidazole-1-carboxylic Acid, 1,1-dimethylethyl Ester

In performing analogously to preparation LXIV, starting with the compound obtained according to preparation XCI, the product sought after is obtained in the form of a white solid (yield=57%).

M.Pt.=88° C.

### EXAMPLE 95

2-[[(2,6-dichlorophenyl)sulphonyl]methylamino]-N-[2-[[[4-(4,5-dihydro-1H-imidazol-2-yl)phenyl]methyl]methylamino]-2-oxoethyl]acetamide, Trifluoroacetate

In performing analogously to Example 1, starting with the compound obtained according to preparation XCII, the product sought after is obtained in the form of a white solid (yield=81%).

M.Pt.=96° C.

Preparation XCIII

γ-oxo-N-[3-(4-pyridinyl)propyl]-1-pyrrolidinebutanamide

In performing analogously to preparation VI, starting with γ-oxo-1-pyrrolidinebutanoic acid and 4-pyridinepropanamine, the product sought after is obtained in the form of a white solid (yield=56%).

M.Pt.=110° C.

Preparation XCIV

N-[4-(1-pyrrolidinyl)butyl]-4-pyridinepropanamine

A solution of 640 mg (2 mmoles) of the compound obtained according to preparation XCIII is prepared in 30 ml of anhydrous tetrahydrofuran and 505 mg (13 mmoles) of lithium aluminium hydride are added. The mixture is agitated under reflux for 20 hours. 20 ml of tetrahydrofuran are then added and then 1 g of hydrated sodium sulphate. The mixture is agitated for 30 minutes at ambient temperature and then filtered. The filtrate is concentrated under reduced pressure and the oily residue is purified by chromatography on C₁₈ grafted silica gel in eluting with the aid of a water/acetonitrile/trifluoroacetic acid mixture (90/10/5; v/v/v). 320 mg of the product sought after are thus obtained in the form of a yellow paste (yield: 20%).

¹H NMR (300 MHz, DMSO) δ: 9.94 (s, 1H); 8.80 (d, 2H); 8.73 (s, 2H); 7.79 (d, 2H); 3.52 (m, 2H); 3.11 (m, 2H); 2.90 (m, 8H); 1.92 (m, 6H); 1.64 (m, 4H).

### EXAMPLE 96

2-[[(2,4-dichloro-3-methylphenyl)sulphonyl]methylamino]-N-[2-oxo-2-[[3-(4-pyridinyl)propyl][4-(1-pyrrolidinyl)butyl]-amino]ethyl]acetamide

In performing analogously to preparation LVI, starting with the acid obtained according to preparation LIX and the amine obtained according to preparation XCIV, the product sought after is obtained in the form of a colourless paste (yield=35%).

¹H NMR (250 MHz, DMSO) δ: 8.45 (m, 2H); 8.02 (t, 1H); 7.88 (d, 1H) 7.63 (d, 1H); 7.25 (m, 2H); 3.99 (s, 2H); 3.94 (t, 2H); 3.25 (m, 4H); 2.88 (s, 3H); 2.56 (m, 2H); 2.50 (s, 3H); 2.40 (m, 6H); 1.85 (m, 2H); 1.65 (s, 4H); 1.41 (m, 4H).

### EXAMPLE 97

2-[[(2,4-dichloro-3-methylphenyl)sulphonyl]methylamino]-N-[2-oxo-2-[[3-(4-pyridinyl)propyl][4-(1-pyrrolidinyl)butyl]amino]ethyl]acetamide, Dihydrochloride

In performing analogously to Example 6, starting with the acid obtained according to Example 96, the product sought after is obtained in the form of a yellow paste (yield=68%).

¹H NMR (300 MHz, DMSO) δ: 10.95 (m, 2H); 8.81 (t, 2H); 8.12 (t, 1H); 7.98 (dd, 2H); 7.89 (d, 1H); 7.65 (d, 1H); 4.00 (s, 4H); 3.96 (t, 2H); 3.49 (m, 2H); 3.33 (m, 4H); 3.07 (m, 2H); 2.91 (m, 2H), 2.87 (s, 3H); 2.49 (s, 3H); 1.92 (m, 6H); 1.62 (m, 4H).

Preparation XCV

4-[[[4-(1-pyrrolidinyl)butyl]amino]methyl]phenol

In performing analogously to preparation XIV, starting with 1-(4-aminobutyl)pyrrolidine and 4-(trimethylsilyloxy)benzaldehyde, the product sought after is obtained in the form of an orange oil (yield=99%)

¹H NMR (300 MHz, DMSO) δ: 7.03 (d, 2H); 6.62 (d, 2H); 3.51 (s, 2H); 2.22.6 (m, 8H); 1.55-1.8 (m, 4H); 1,3-1.5 (m, 4H).

### EXAMPLE 98

2-[[(2,4-dichloro-3-methylphenyl)sulphonyl]methylamino]-N-[2-[[(4-hydroxyphenyl)methyl][4-(1-pyrrolidinyl)butyl]amino]-2-oxoethyl]acetamide

In performing analogously to Example 96, starting with the amine obtained according to preparation XCV, the product sought after is obtained in the form of a white solid (yield=33%).

M.Pt.=104° C.

Preparation XCVI

N-[2-[[(2-chlorophenyl)sulphonyl]methylamino]acetyl]glycine, Ethyl Ester

In performing analogously to preparation LXXXIX, starting with N-methyl-2-chlorobenzenesulphonamide, the product sought after is obtained, and is used without further purification for the next synthesis.

Preparation XCVII

N-[2-[[(2-chlorophenyl)sulphonyl]methylamino]acetyl]glycine

In performing analogously to preparation LIX, starting with the compound obtained according to preparation XCVI, the product sought after is obtained in the form of a beige solid (yield=74%).

M.Pt.=132° C.

Preparation XCVIII

2-[4-[[[2-[[2-[[(2-chlorophenyl)sulphonyl]methylamino]acetyl]amino]acetyl]methylamino]methyl]phenyl]-4,5-dihydro-1H-imidazole-1-carboxylic acid, 1,1-dimethylethyl Ester

In performing analogously to preparation XCII, starting with the compound obtained according to preparation XCVII, the product sought after is obtained in the form of a white solid (yield=45%).

M.Pt.=82° C.

### EXAMPLE 99

2-[[(2-chlorophenyl)sulphonyl]methylamino]-N-[2-[[[4-(4,5-dihydro-1H-imidazol-2-yl)phenyl]methyl]methylamino]-2-oxoethyl]acetamide, Trifluoroacetate

In performing analogously to Example 1, starting with the compound obtained according to preparation XCVIII, the product sought after is obtained in the form of a white solid (yield=80%).

M.Pt.=100° C.

Preparation IC

2-[4-[[[2-[[2-[[(2,3,4-trichlorophenyl) sulphonyl]methylamino]acetyl]amino]acetyl]methylamino]methyl]phenyl]-4,5-dihydro-1H-imidazole-1-carboxylic Acid, 1,1-dimethylethyl Ester

In performing analogously to preparation XCII, starting with the compound obtained according to preparation LXXXIII, the product sought after is obtained in the form of a white solid (yield=67%).

M.Pt.=94° C.

### EXAMPLE 100

2-[[(2,3,4-trichlorophenyl)sulphonyl]methylamino]-N-[2-[[[4-(4,5-dihydro-1H-imidazol-2-yl)phenyl]methyl]methylamino]-2-oxoethyl]acetamide, Trifluoroacetate

In performing analogously to Example 1, starting with the compound obtained according to preparation IC, the product sought after is obtained in the form of a white solid (yield=60%).

M.Pt.=95° C.

Preparation C

2-[[(2,4-dichloro-3-methylphenyl)sulphonyl]amino]-N-methylacetamide

In performing analogously to preparation I, starting with 2-amino-N-methylacetamide, the product sought after is obtained in the form of a white solid (yield=76%).

M.Pt.=148° C.

Preparation CI

N-[2-[[(2,4-dichloro-3-methylphenyl)sulphonyl][2-(methylamino)-2-oxoethyl]amino]acetyl]glycine, Ethyl Ester

In performing analogously to preparation II, starting with the compound obtained according to preparation C, the product sought after is obtained in the form of a white solid (yield=63%).

M.Pt.=140° C.

Preparation CII

N-[2-[[(2,4-dichloro-3-methylphenyl)sulphonyl][2-(methylamino)-2-oxo-ethyl]amino]acetyl]glycine

In performing analogously to preparation LIX, starting with the compound obtained according to preparation CI, the product sought after is obtained in the form of a white ecru solid (yield=81%).

M.Pt.=205° C.

Preparation CIII

2-[4-[8-[(2,4-dichloro-3-methylphenyl)sulphonyl]-2-methyl-3,6, 10-trioxo-2,5,8,11-tetraazadodec-1-yl]phenyl]-4,5-dihydro-1H-imidazole-1-carboxylic Acid, 1,1-dimethylethyl Ester

In performing analogously to preparation XCII, starting with the compound obtained according to preparation CII, the product sought after is obtained in the form of a white solid (yield=53%).

M.Pt.=105° C.

### EXAMPLE 101

2-[[(2,4-dichloro-3-methylphenyl)sulphonyl][2-[[2-[[[4-(4,5-dihydro-1H-imidazol-2-yl)phenyl]methyl]methylamino]-2-oxoethyl]amino]-2-oxo-ethyl]amino]-N-methyl-acetamide, Trifluoroacetate

In performing analogously to Example 1, starting with the compound obtained according to preparation CIII, the product sought after is obtained in the form of a white solid (yield=99%).

M.Pt.=106° C.

Preparation CIV

2-[4-[[[2-[[2-[[(2,4-dichloro-3-methylphenyl)sulphonyl]-2-propenylamino]acetyl]amino]acetyl]methylamino]methyl]phenyl]-4,5-dihydro-1H-imidazole-1-carboxylic Acid, 1,1-dimethylethyl Ester

In performing analogously to preparation XCII, starting with the compound obtained according to preparation LXXXVII, the product sought after is obtained in the form of a white solid (yield=35%).

M.Pt.=70° C.

### EXAMPLE 102

2-[[(2,4-dichloro-3-methylphenyl)sulphonyl]2-propenylamino]-N-[2-[[[4-(4,5-dihydro-1H-imidazol-2-yl)phenyl]methyl]methylamin]-2-xo-ethyl]acetamide

In performing analogously to Example 1 and by adding aqueous ammonia, starting with the compound obtained according to preparation CIV, the product sought after is obtained in the form of a white solid (yield=84%).

M.Pt.=90° C.

### EXAMPLE 103

2-[[(2,4-dichloro-3-methylphenyl)sulphonyl]2-propenylamino]-N-[2-[[[4-(4,5-dihydro-1H-imidazol-2-yl)phenyl]methyl]methylamino]-2-oxo-ethyl]acetamide, Hydrochloride

In performing analogously to Example 6, starting with the compound obtained according to Example 102, the product sought after is obtained in the form of a white solid (yield=54%).

M.Pt.=125° C.

Preparation CV

2-[4-[8-[(2,4-dichloro-3-methylphenyl)sulphonyl]-2-methyl-3,6-dioxo-11-oxa-2,5,8-triazadodec-1-yl]phenyl]-4,5-dihydro-1H-imidazole-1-carboxylic Acid, 1,1-dimethylethyl Ester

In performing analogously to preparation XCII, starting with the acid obtained according to preparation LXXI, the product sought after is obtained in the form of a white solid (yield=76%).

M.Pt.=80° C.

### EXAMPLE 104

2-[[(2,4-dichloro-3-methylphenyl)sulphonyl](2-methoxyethyl)amino]-N-[2-[[[4-(4,5-dihydro-1H-imidazol-2-yl)phenyl]methyl]methylamino]-2-oxoethyl]acetamide

In performing analogously to Example 102, starting with the compound obtained according to preparation CV, the product sought after is obtained in the form of a white ecru solid (yield=99%).

M.Pt.=76° C.

### EXAMPLE 105

2-[[(2,4-dichloro-3-methylphenyl)sulphonyl](2-methoxyethyl)amino]-N-[2-[[[4-(4,5-dihydro-1H-imidazol-2-yl)phenyl]methyl]methylamino]-2-xethyl]acetamide, Hydr chloride

In performing analogously to Example 6, starting with the compound obtained according to Example 104, the product sought after is obtained in the form of a white solid (yield=85%).

M.Pt.=130° C.

Preparation CVI

N-methyl-2,3-dichlorobenzenesulphonamide

In performing analogously to preparation LVII, starting with 2,3-dichlorobenzenesulphonyl chloride, the product sought after is obtained which is used without further purification in the next synthesis.

Preparation CVII

N-2-[[(2,3-dichlorophenyl)sulphonyl]methylamino]acetyl]glycine, Ethyl Ester

In performing analogously to preparation LXXXII, starting with the compound obtained according to preparation CVI, the product sought after is obtained which is used without further purification in the next synthesis.

Preparation CVIII

N-2-[[(2,3-dichlorophenyl)sulphonyl]methylamino]acetyl]glycine

In performing analogously to preparation LIX, starting with the compound obtained according to preparation CVII, the product sought after is obtained in the form of a white solid (yield=55%).

M.Pt.=147° C.

Preparation CIX

2-[4-[[[2-[[2-[[(2,3-dichlorophenyl)sulphonyl]methylamino]acetyl]-amino]acetyl]methylamino]methyl]phenyl]-4,5-dihydro-1H-imidazole-1-carboxylic Acid, 1,1-dimethylethyl Ester

In performing analogously to preparation XCII, starting with the compound obtained according to preparation CVIII, the product sought after is obtained in the form of a white solid (yield=59%).

M.Pt.=88° C.

### EXAMPLE 106

2-[[(2,3-dichlorophenyl)sulphonyl]methylamino]-N-[2-[[[4,5-dihydro-1H-imidazole-2-yl)phenyl]methyl]methylamino]2-oxoethyl]acetamide, Trifluoroacetate

In performing analogously to Example 1, starting with the compound obtained according to preparation CIX, the product sought after is obtained in the form of a white solid (yield=80%).

M.Pt.=100° C.

Preparation CX

N-[2-[[(2,4-dichlorophenyl)sulphonyl]methylamino]acetyl]glycine

In performing analogously to preparations LXXXIX to XCI, starting with 2,4-dichlorobenzenesulphonyl chloride, the product sought after is obtained in the form of a white solid (yield=30%).

M.Pt.=179° C.

Preparation CXI

2-[4-[[[2-[[2-[[(2,4-dichlorophenyl)sulphonyl]methylamino]acetyl]amino]acetyl]methylamino]methyl]phenyl]-4,5-dihydro-1H-imidazole-1-carboxylic Acid, 1,1-dimethylethyl Ester

In performing analogously to preparation LXIV, starting with the compound obtained according to preparation CX, the product sought after is obtained in the form of a white solid (yield=68%).

M.Pt.=72° C.

### EXAMPLE 107

2-[[(2,4-dichlorophenyl)sulphonyl]methylamino]-N-[2-[[[4-(4,5-dihydro-1H-imidazol-2-yl)phenyl]methyl]methylamino]-2-oxoethyl]acetamide, Trifluoroacetate

In performing analogously to Example 1, starting with the compound obtained according to preparation CXI, the product sought after is obtained in the form of a white solid (yield=99%).

M.Pt.=90° C.

### EXAMPLE 108

2-[[(2,4-dichlorophenyl)sulphonyl]methylamino]-N-[2-[methyl[4-(1-pyrrolidinyl)butyl]amino]-2-oxoethyl]acetamide

In performing analogously to preparation LVI, starting with the acid obtained according to preparation LIX and N-methyl-1-pyrrolidinebutanamide, the product sought after is obtained in the form of a yellow oil (yield=91%).

¹H NMR (300 MHz, DMSO) δ: 8.01 (m, 1H); 7.89 (d, 1H); 7.64 (d, 1H); 3.99 (s, 2H); 3.94 (m, 2H); 3.26 (m, 6H); 2.91 (s) and 2.80 (s) (total 3H); 2.88 (s, 3H); 2.50 s, 3H); 2.38 (m, 2H); 1.65 (s, 4H); 1.44 (m, 4H).

### EXAMPLE 109

2-[[(2,4-dichlorophenyl)sulphonyl]methylamino]-N-[2-[methyl[4-(1-pyrrolidinyl)butyl]amino]-2-oxoethyl]acetamide, Fumarate

A solution of 127 mg (0.25 mM) of the compound obtained according to Example 108 is prepared in 6 ml of methanol and 29 mg (0.25 mM) of fumaric acid are added. The reaction mixture is agitated for 15 min and then concentrated under reduced pressure. The residue is dissolved in 10 ml of water and then lyophilised. 145 mg the product sought after are thus obtained in the form of an amorphous solid (yield=93%).

¹H NMR (250 MHz, DMSO) δ: 8.04 (m, 1H); 8.01 (d, 1H); 7.64 (d, 1H); 5.51 (s, 2H); 4.00 (s, 2H); 3.95 (t, 2H); 3.28 (m, 2H); 2.87 (m, 12H); 2.49 (s, 3H); 1.81 (s, 4H); 1.49 (m, 4H).

Preparation CXII

N-[2-[methyl(1-naphthalenylsulphonyl)amino]acetyl]glycine In performing analogously to preparation CX, starting with 1-naphthalenesulphonyl chloride, the product sought after is obtained in the form of a yellow solid (yield=40%).

M.Pt.=120° C.

Preparation CXIII

4,5-dihydro-2-[4-[[methyl[2-[[2-[methyl(1-naphthalenylsulphonyl)amino]acetyl]amino]methyl]phenyl]-1H-imidazole-1-carboxylic Acid, 1,1-dimethylethyl Ester

In performing analogously to preparation XCII, starting with the compound obtained according to preparation CXII, the product sought after is obtained in the form of a white solid (yield=53%).

M.Pt.=90° C.

### EXAMPLE 110

N-[2-[[[4-(4,5-dihydro-1H-imidazol-2-yl)phenyl]methyl]methylamino]-2-oxoethyl]-2-[methyl(1-naphthalenylsulphonyl)amino]acetamide, Trifluoroacetate

In performing analogously to Example 1, starting with the compound obtained according to preparation CXIII, the product sought after is obtained in the form of a white solid (yield=88%).

M.Pt.=115° C.

Preparation CXIV

N-[2-[methyl(2-naphthalenylsulphonyl)amino]acetyl]glycine

In performing analogously to preparation CX, starting with 2-naphthalenesulphonyl chloride, the product sought after is obtained in the form of a white solid (yield=54%).

M.Pt.=185° C.

Preparation CXV

4,5-dihydro-2-[4-[[methyl[2-[[2-[methyl(2-naphthalenylsulphonyl)amino]acetyl]amino]methyl]phenyl]-1H-imidazole-1-carboxylic Acid, 1,1-dimethylethyl Ester

In performing analogously to preparation XCII, starting with the compound obtained according to preparation CXIV, the product sought after is obtained in the form of a white solid (yield=62%).

M.Pt.=89° C.

### EXAMPLE 111

N-[2-[[[4-(4,5-dihydro-1H-imidazol-2-yl)phenyl]methyl]methylamino]-2-oxoethyl]-2-[methyl(2-naphthalenylsulphonyl)amino]acetamide, Tri-fluoroacetate

In performing analogously to Example 1, starting with the compound obtained according to preparation CXV, the product sought after is obtained in the form of a white solid (yield=85%).

M.Pt.=101° C.

Preparation CXVI

N-cyclopropyl-2,6-dichlorobenzenesulphonamide

In performing analogously to preparation LXXXIX, starting with cyclopropanamine, the product sought after is obtained in the form of a white solid (yield=99%).

M.Pt.=76° C.

Preparation CXVII

N-[2-[cyclopropyl[(2,6-dichlorophenyl)sulphonyl]amino]acetyl]glycine, Ethyl Ester

In performing analogously to preparation XC, starting with the compound obtained according to preparation CXVI, the product sought after is obtained in the form of a yellow solid (yield=76%).

M.Pt.=125° C.

Preparation CXVIII

N-[2-[cyclopropyl[(2,6-dichlorophenyl)sulphonyl]amino]acetyl]glycine

In performing analogously to preparation XCI, starting with the compound obtained according to preparation CXVII, the product sought after is obtained in the form of a white solid (yield=62%).

M.Pt.=164° C.

Preparation CXIX

2-[4-[[[2-[[2-[cyclopropyl[(2,6-dichlorophenyl)sulphonyl]amino]acetyl]amino]acetyl]methylamino]methyl]phenyl]-4,5-dihydro-1H-imidazole-1-carboxylic Acid, 1,1-dimethylethyl Ester

In performing analogously to preparation XCII, starting with the compound obtained according to preparation CXVIII, the product sought after is obtained in the form of a white solid (yield=55%).

M.Pt.=66° C.

### EXAMPLE 112

2-[cyclopropyl[(2,6-dichlorophenyl)sulphonyl]amino]-N-[2-[[[4-(4,5-dihydro-1H-imidazol-2-yl)phenyl]methyl]methylamino]-2-oxoethyl]-acetamide, Trifluoroacetate

In performing analogously to Example 1, starting with the compound obtained according to preparation CXIX, the product sought after is obtained in the form of a white solid (yield=71%).

M.Pt.=118° C.

Preparation CXX

N-cyclopropyl-2,3-dichlorobenzenesulphonamide In performing analogously to preparation CVI, starting with cyclopropanamine, the product sought after is obtained in the form of a white ecru solid (yield=50%).

M.Pt.=140° C.

Preparation CXXI

N-[2-[cyclopropyl[(2,3-dichlorophenyl)sulphonyl]amino]acetyl]glycine, Ethyl Ester

In performing analogously to preparation XC, starting with the compound obtained according to preparation CXX, the product sought after is obtained in the form of a white solid (yield=89%).

M.Pt.=155° C.

Preparation CXXII

N-[2-[cyclopropyl[(2,3-dichlorophenyl)sulphonyl]amino]acetyl]glycine

In performing analogously to preparation XCI, starting with the compound obtained according to preparation CXXI, the product sought after is obtained in the form of a white ecru solid (yield=72%).

M.Pt.=174° C.

Preparation CXXIII

2-[4-[[[2-[[2-[cyclopropyl[(2,3-dichlorophenyl)sulphonyl]amino]acetyl]amino]acetyl]methylamino]methyl]phenyl]-4,5-dihydro-1H-imidazole-1-carboxylic Acid, 1,1-dimethylethyl Ester

In performing analogously to preparation XCII, starting with the compound obtained according to preparation CXXII, the product sought after is obtained in the form of a white solid (yield=67%).

M.Pt.=98° C.

### EXAMPLE 113

2-[cyclopropyl[(2,3-dichlorophenyl)sulphonyl]amino]-N-[2-[[[4-(4,5-dihydro-1H-imidazol-2-yl)phenyl]methyl]methylamino]-2-oxoethyl]acetamide, Trifluoroacetate

In performing analogously to Example 1, starting with the compound obtained according to preparation CXXIII, the product sought after is obtained in the form of a white solid (yield=84%).

M.Pt.=88° C.

Preparation CXXIV

2-chloro-N-cyclopropyl-benzenesulphonamide

In performing analogously to preparation CXVI, starting with 2-chlorobenzenesulphonyl chloride, the product sought after is obtained in the form of a white solid (yield=82%).

M.Pt.=117° C.

Preparation CXXV

N-[2-[[(2-chlorophenyl)sulphonyl]cyclopropylamino]acetyl]glycine, Ethyl Ester

In performing analogously to preparation CXXIV, starting with the compound obtained according to preparation CXXIV, the product sought after is obtained in the form of a white solid (yield=93%).

M.Pt.=98° C.

Preparation CXXVI

N-[2-[[(2-chlorophenyl)sulphonyl]cyclopropylamino]acetyl]glycine

In performing analogously to preparation XCI, starting with the compound obtained according to preparation CXXV, the product sought after is obtained in the form of a yellow solid (yield=72%).

M.Pt.=125° C.

Preparation CXXVII

2-[4-[[[2-[[2-[[(2-chlorophenyl)sulponyl]cyclopropylamino]acetyl]amino]acetyl]methylamino]methyl]-phenyl]-4,5-dihydro-1H-imidazole-1-carboxylic Acid, 1,1-dimethylethyl Ester

In performing analogously to preparation CXII, starting with the compound obtained according to preparation CXXVI, the product sought after is obtained in the form of a white solid (yield=75%).

M.Pt.=70° C.

### EXAMPLE 114

2-[[(2-chlorophenyl)sulphonyl]cyclopropylamino]-N-[2-[[[4-(4,5-dihydro-1H-imidazol-2-yl)phenyl]methyl]methylamino]-2-oxoethyl]acetamide, Trifluoroacetate

In performing analogously to Example 1, starting with the compound obtained according to preparation CXXVII, the product sought after is obtained in the form of a white solid (yield=76%).

M.Pt.=106° C.

Preparation CXXVIII

2-[[(2,6-dichlorophenyl)sulphonyl]amino]acetamide

In performing analogously to preparation CXVI, starting with 2-aminoacetamide, the product sought after is obtained in the form of a white solid (yield=54%).

M.Pt.=164° C.

Preparation CXXIX

N-[2-[(2-amino-2-oxoethyl)[(2,6-dichlorophenyl)sulphonyl]amino]acetyl]glycine, Ethyl Ester

In performing analogously to preparation II, starting with the compound obtained according to preparation CXXVIII, the product sought after is obtained in the form of a beige solid (yield=31%).

M.Pt.=178° C.

Preparation CXXX

N-[2-[(2-amino-2-oxoethyl)[(2,6-dichlorophenyl)sulphonyl]amino]acetyl]glycine,

In performing analogously to preparation LXXV, starting with the compound obtained according to preparation CXXIX, the product sought after is obtained in the form of a beige paste (yield=99%).

¹H NMR (250 MHz, DMSO) δ: 8.61 (t, 1H); 7.68 (s, 1H); 7.61 (m, 2H) 7.52 (dd, 1H); 7.10 (s, 1H); 4.21 (s, 2H); 4.08 (s, 2H); 3.74 (d, 2H).

Preparation CXXXI

2-[4-[[[2-[[2-[(2-amino-2-oxoethyl)[(2,6-dichlorophenyl)sulphonyl]amino]acetyl]amino]acetyl]methylamino]methyl]phenyl]-4,5-dihydro-1H-imidazole-1-carboxylic Acid, 1,1-dimethylethyl Ester

In performing analogously to preparation VI, starting with the compounds obtained according to preparations CXXX and LXIII, the product sought after is obtained in the form of a colourless paste (yield=15%).

¹H NMR (300 MHz, CDCl₃) δ: 7.47 (m, 5H); 7.32 (m, 2H); 7.21 (m, 2H) 5.75 (s, 1H); 4.61 (s, 2H); 4.26 (s, 2H); 4.1 (m, 4H); 3.96 (m, 4H); 2.87 (s, 3H); 1.27 (s, 9H).

### EXAMPLE 115

2-[(2-amino-2-oxoethyl)[(2,6-dichlorophenyl)sulphonyl]amino]-N-[2-[[[4-(4,5-dihydro-1H-imidazol-2-yl)phenyl]methyl]methylamino]-2-oxoethyl]acetamide, Trifluoroacetate

In performing analogously to Example 1, starting with the compound obtained according to preparation CXXXI, the product sought after is obtained in the form of a white solid (yield=90%).

M.Pt.=124° C.

Preparation CXXXII

2-[[(2,3-dichlorophenyl)sulphonyl]amino]acetamide

In performing analogously to preparation CVI, starting with 2-aminoacetamide, the product sought after is obtained in the form of a white solid (yield=54%).

M.Pt.=152° C.

Preparation CXXXIII

N-[2-[(2-amino-2-oxoethyl)[(2,3-dichlorophenyl)sulphonyl]amino]-acetyl]glycine, Ethyl Ester

In performing analogously to preparation II, starting with the compound obtained according to preparation CXXXII, the product sought after is obtained in the form of a beige solid (yield=46%).

M.Pt.=208° C.

Preparation CXXXIV

N-[2-[(2-amino-2-oxoethyl)[(2,3-dichlorophenyl)sulphonyl]amino]acetyl]glycine,

In performing analogously to preparation LXXV, starting with the compound obtained according to preparation CXXXIII, the product sought after is obtained in the form of a beige solid (yield=99%).

M.Pt.=110° C.

Preparation CXXXV

2-[4-[[[2-[[2-[(2-amino-2-oxoethyl)[(2.3-dichlorophenyl)sulphonyl]amino]acetyl]amino]acetyl]methylamino]methyl]phenyl]-4,5-dihydro-1H-imidazole-1-carboxylic Acid, 1,1-dimethylethyl Ester

In performing analogously to preparation VI, starting with the compounds obtained according to preparations CXXXIV and LXIII, the product sought after is obtained in the form of a white solid (yield=64%).

M.Pt.=118° C.

### EXAMPLE 116

2-[(2-amino-2-oxoethyl)[(2,3-dichlorophenyl)sulphonyl]amino]-N-[2-[[[4-(4,5-dihydro-1H-imidazol-2-yl)phenyl]methyl]methylamino]-2-oxo-ethyl]acetamide, Trifluoroacetate

In performing analogously to Example 1, starting with the compound obtained according to preparation CXXXV, the product sought after is obtained in the form of a white solid (yield=71%).

M.Pt.=122° C.

The chemical structures of the compounds of the invention described above are listed in Table 1 in the case in which R represents a phenylethyl group, in Table 2 in the case in which R represents a hydrogen atom or a methyl group, and in Table 3 in the case in which the X substituent is different from a methyl group.

TABLE I

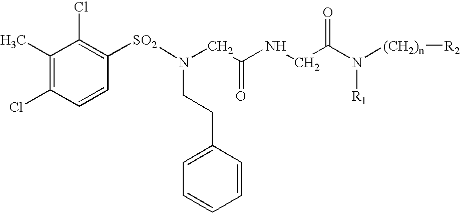

| Ex | R₁ | R₂ | n | Salt |
|---|---|---|---|---|
| 1 | —(CH₂)₄—NH₂ | 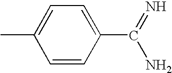 | 1 | TFA |
| 2 | —(CH₂)₃—NH₂ | 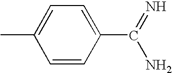 | 1 | TFA |
| 3 | 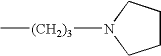 | 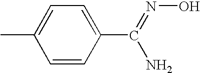 | 1 | — |
| 4 | 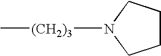 | 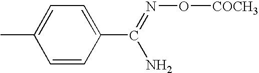 | 1 | — |
| 5 | 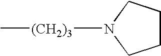 | 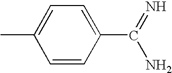 | 1 | — |
| 6 | 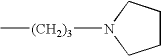 | 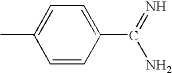 | 1 | 2HCl |
| 7 | 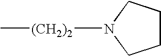 | 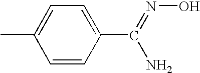 | 1 | — |
| 8 | 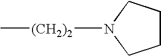 | 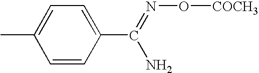 | 1 | — |
| 9 | 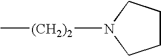 | 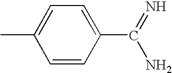 | 1 | — |
| 10 | 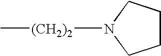 | 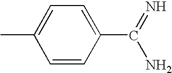 | 1 | 2HCl |
| 11 | 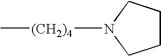 | 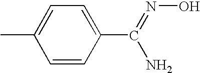 | 1 | — |
| 12 | 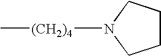 | 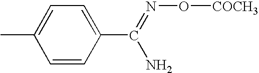 | 1 | — |
| 13 | 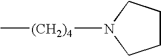 | 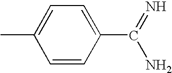 | 1 | — |
| 14 | 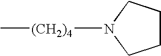 | 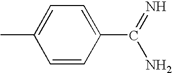 | 1 | 2HCl |
| 15 | —(CH₂)₄—N(CH₃)₂ | 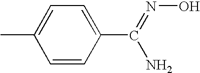 | 1 | — |
| 16 | —(CH₂)₄—N(CH₃)₂ | 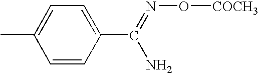 | 1 | — |
| 17 | —(CH₂)₄—N(CH₃)₂ | 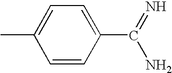 | 1 | — |
| 18 | —(CH₂)₄—N(CH₃)₂ | 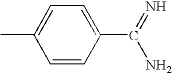 | 1 | 2HCl |
| 19 | —(CH₂)₄—N(CH₃)₂ | 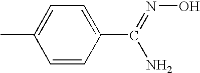 | 1 | |
| 20 | —(CH₂)₄—N(CH₃)₂ | 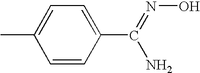 | 1 | 2HCl |
| 21 | —(CH₂)₄—N(CH₃)₂ | 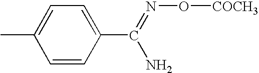 | 1 | |
| 22 | —(CH₂)₄—N(CH₃)₂ | 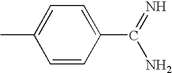 | 1 | |
| 23 | —(CH₂)₄—N(CH₃)₂ | 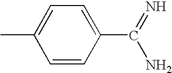 | 1 | 2HCl |
| 24 | 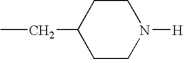 | 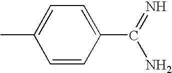 | 1 | 2TFA |
| 25 | —(CH₂)₄—NH₂ | 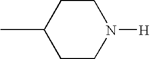 | 1 | 2TFA |
| 26 | —(CH₂)₄—OH | 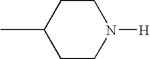 | 1 | 1TFA |
| 27 | —(CH₂)₄—NH—COCH₃ | 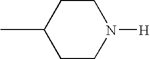 | 1 | 1 TFA |
| 28 | 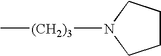 | 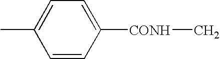 | 1 | — |
| 29 | 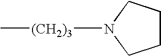 | 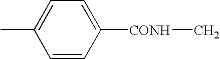 | 1 | 1HCl |
| 30 | 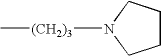 | 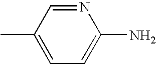 | 1 | |
| 31 | 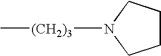 | 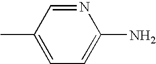 | 1 | 2HCl |
| 32 | —(CH₂)₃—N(CH₃)₂ | —CH₂—N(CH₃)₂ | 2 | |
| 33 | —(CH₂)₃—N(CH₃)₂ | —(CH₂)₃—N(CH₃)₂ | 2 | 2HCl |
| 34 | —(CH₂)₃—N(CH₃)₂ | 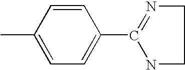 | 1 | |
| 35 | —(CH₂)₃—N(CH₃)₂ | 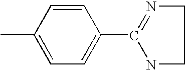 | 1 | 2HCl |
| 36 | —CH₃ | 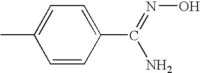 | 1 | |
| 37 | —CH₃ | 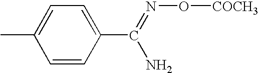 | 1 | |
| 38 | —CH₃ | 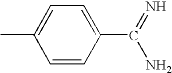 | 1 | |
| 39 | —CH₃ | 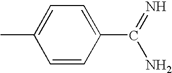 | 1 | 1HCl |
| 40 | —CH₃ | 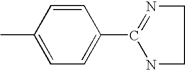 | 1 | |
| 41 | —CH₃ | 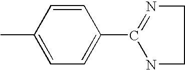 | 1 | 1HCl |
| 42 | H | 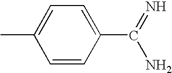 | 1 | |
| 43 | H | 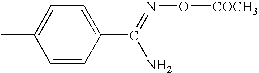 | 1 | |
| 44 | H | 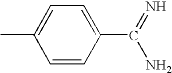 | 1 | |
| 45 | H | 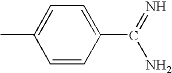 | 1 | 1HCl |
| 46 | 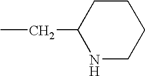 | 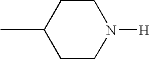 | 1 | 2TFA |
| 47 | 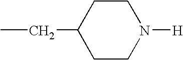 | 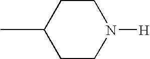 | 1 | 2TFA |
| 48 | 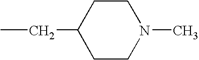 | 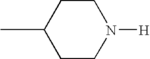 | 1 | 2TFA |
| 49 | 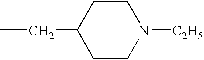 | 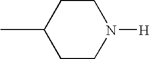 | 1 | 2TFA |
| 50 | 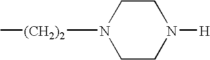 | 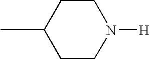 | 1 | 2TFA |
| 51 | 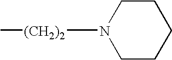 | 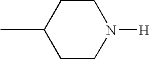 | 1 | 2TFA |
| 52 | 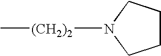 | 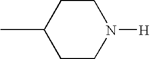 | 1 | 2TFA |
| 53 | 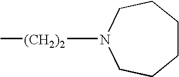 | 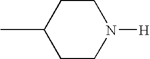 | 1 | 2TFA |
| 54 | 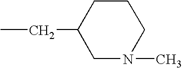 | 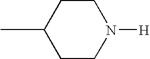 | 1 | 2TFA |
| 55 | 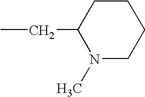 | 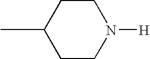 | 1 | 2TFA |
| 56 | —(CH₂)₃—NH₂ | 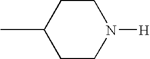 | 1 | 2TFA |
| 57 | —(CH₂)₃—N(CH₃)₂ | 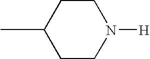 | 1 | 2TFA |
| 58 | 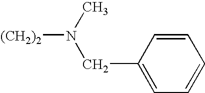 | 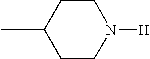 | 1 | 2TFA |
| 59 | 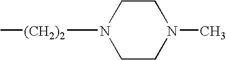 | 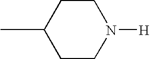 | 1 | 2TFA |
| 60 | 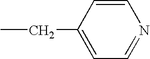 | 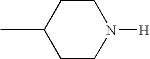 | 1 | 2TFA |
| 61 | —(CH₂)₂—N(CH₃)₂ | —CH₂—NH₂ | 3 | 2TFA |
| 62 | —(CH₂)₃—N(CH₃)₂ | —CH₂—NH₂ | 3 | 2TFA |
| 63 | 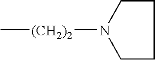 | —CH₂—NH₂ | 3 | 2TFA |
| 64 | 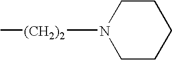 | —CH₂—NH₂ | 3 | 2TFA |
| 65 | 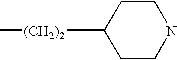 | —CH₂—NH₂ | 3 | 2TFA |
| 66 | 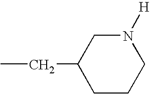 | —CH₂—NH₂ | 3 | 2TFA |
| 67 | 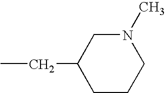 | —CH₂—NH₂ | 3 | 2TFA |
| 68 | 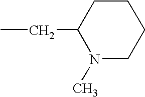 | —CH₂—NH₂ | 3 | 2TFA |
| 69 | 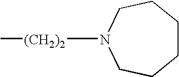 | —CH₂—NH₂ | 3 | 2TFA |
| 70 | 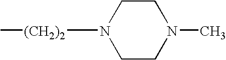 | —CH₂—NH₂ | 3 | 2TFA |
| 71 | 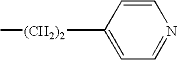 | —CH₂—NH₂ | 3 | 2TFA |
| 72 | 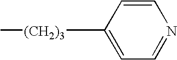 | —CH₂—NH₂ | 3 | 2TFA |
| 73 | 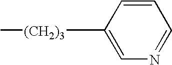 | —CH₂—NH₂ | 3 | 2TFA |
| 74 | 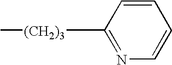 | —CH₂—NH₂ | 3 | 2TFA |
| 75 | 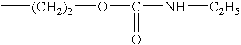 | —CH₂—NH₂ | 3 | 1TFA |
| 76 | 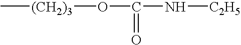 | —CH₂—NH₂ | 3 | 1TFA |
| 77 | 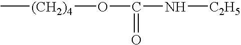 | —CH₂—NH₂ | 3 | 1TFA |
| 78 | 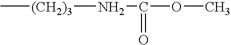 | —CH₂—NH₂ | 3 | 1TFA |
| 79 | 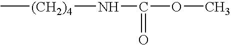 | —CH₂—NH₂ | 3 | 1TFA |
| 80 | —(CH₂)₃—NH₂ | —CH₂—NH₂ | 3 | 2TFA |
| 81 | 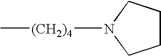 | 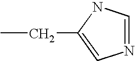 | 2 | |
| 82 | 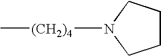 | 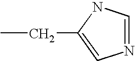 | 2 | 2HCl |

TABLE 2

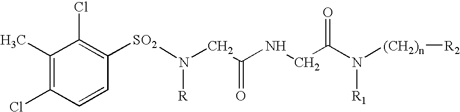

| Ex | R | R₁ | R₂ | n | Salt |
|---|---|---|---|---|---|
| 83 | CH₃ | CH₃ | 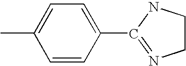 | 1 | |
| 84 | CH₃ | CH₃ | 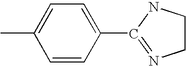 | 1 | HCl |
| 85 | H | 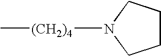 | 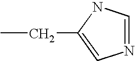 | 2 | — |
| 86 | H | 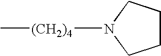 | 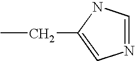 | 2 | 2HCl |
| 87 | —CH₂—CH₂—O—CH₃ | 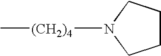 | 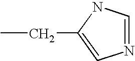 | 2 | — |
| 88 | —CH₂—CH₂—O—CH₃ | 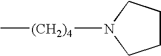 | 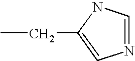 | 2 | 2 HCl |
| 89 | 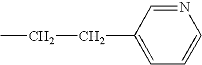 | 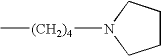 | 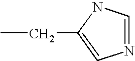 | 2 | — |
| 90 | 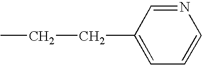 | 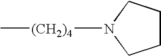 | 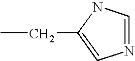 | 2 | 3TFA |
| 91 | —CH₂—CO—NH₂ | CH₃ | 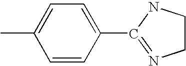 | 1 | TFA |
| 93 | —CH₂—CH═CH₂ | 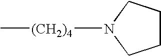 | 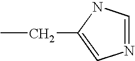 | 2 | — |
| 94 | —CH₂—CH═CH₂ | 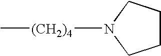 | 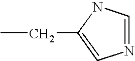 | 2 | 2HCl |
| 96 | CH₃ | 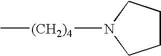 | 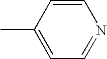 | 3 | — |
| 97 | CH₃ | 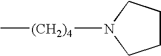 | 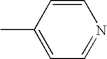 | 3 | 2HCl |
| 98 | CH₃ | 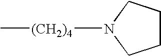 | 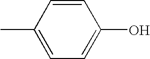 | 1 | — |
| 101 | —CH₂—CO—NH—CH₃ | CH₃ | 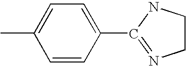 | 1 | TFA |
| 102 | —CH₂—CH═CH₂ | CH₃ | 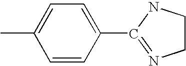 | 1 | — |
| 103 | —CH₂—CH═CH₂ | CH₃ | 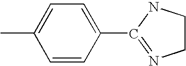 | 1 | HCl |
| 104 | CH₂—CH₂—O—CH₃ | CH₃ | 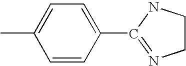 | 1 | — |
| 105 | CH₂CH₂—O—CH₃ | CH₃ | 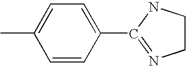 | 1 | HCl |
| 108 | CH₃ | CH₃ | 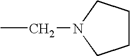 | 3 | — |
| 109 | CH₃ | CH₃ | 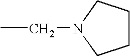 | 3 | Fumarate |

TABLE 3

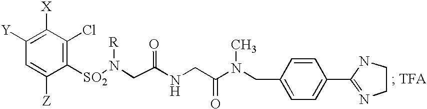

| Ex: | X | Y | Z | R |
|---|---|---|---|---|
| 95 | H | H | Cl | CH₃ |
| 99 | H | H | H | CH₃ |
| 100 | Cl | Cl | Cl | CH₃ |
| 106 | Cl | H | H | CH₃ |
| 107 | H | Cl | H | CH₃ |
| 112 | H | H | Cl | 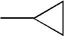 |
| 113 | Cl | H | H | 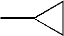 |
| 114 | H | H | H | 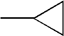 |
| 115 | H | H | Cl | CH₂CONH₂ |
| 116 | Cl | H | H | CH₂CONH₂ |

Note:
all compounds cited in Table 3 are in the form of a salt with trifluoroacetic acid.

Biological Activity

The compounds of the present invention were evaluated for their analgesic property in the formaldehyde-induced pain test in the mouse (Shibata, M., Ohkubo, T., Takahashi, H. & R. Inoki. Modified formalin test: characteristic biphasic pain response. Pain, 38. 347-352). In summary, an administration of formaldehyde (0.92% in physiological serum) is made in the front paw and the licking time, which reflects the intensity of the pain, is recorded from 0 to 5 min (1^{st} phase) and from 15 to 30 min (2^{nd} phase) after the injection. The percentage inhibition of the second phase of licking induced by the formaldehyde is given, for some compounds according to the invention, in the following Table:

| | Dose | Administration | % inhibition of the |
|---|---|---|---|
| Example | (mg/kg) | route | 2^{nd} licking phase |
| 14 | 10 | i.p. | 93 |
| 82 | 10 | i.p. | 63 |
| 84 | 30 | i.p; | 57 |
| 25 | 30 | s.c. | 87 |
| 23 | 30 | s.c. | 98 |
| 24 | 10 | s.c. | 76 |
| 35 | 10 | s.c. | 92 |

i.p. intraperitoneal
s.c. sub-cutaneous

These results show a very significant decrease in the pain after administration of the compounds.

Further to the results of the previous test, the compounds according to the invention were subjected to a test aiming to demonstrate their mode of action and making use of the bradykinin B1 receptor.

This test makes use of the human umbilical vein and is carried out according to the following protocol:

Human umbilical cords of 15-25 cm in length are recovered just after delivery and were immediately placed in a flask containing a Krebs solution of composition (in mM): NaCl 119, KCl 4.7, KH₂PO₄ 1.18, MgSO₄ 1.17, NaHCO₃ 25, CaCl₂ 2.5, glucose 5.5, EDTA 0.026, and were then stored at 4° C.

The cord is dissected under Krebs solution so as to spread out the umbilical cord. The vein is cleaned of any adhering tissue and is cut into small rings of 3-4 mm wide. The endothelium is carefully removed by introduction of a fine catheter No. 1, rendered slightly abrasive, into the lumen of the vessel.

In order to induce the expression of the bradykinin B₁ receptor, the vein segments are incubated at 37° C. in a 25 ml container for 16 hours in an EMEM culture medium which is oxygenated by a 95% O₂+5% CO₂ mixture to which medium the antibiotics: penicillin 10000 IU/ml and streptomycin 10000 GU/ml, are added.

The next day, the vein rings are mounted on a stainless steel support linked to an isometric sensor and are placed in an 8 ml isolated organ container thermostated at 37° C., containing a Krebs solution oxygenated by a 95% O₂+5% CO₂ mixture.

After a rest period of one hour during which the rings are rinsed 5 to 6 times with a Krebs solution (kept at 37° C. throughout the whole manipulation and oxygenated by the 95% O₂+5% CO₂ mixture), the vein is subjected progressively to a tension of 1 g. When the tension is stable, after 45 about minutes, the Krebs solution is replaced with a hyperpotassium solution (KPSS: at a temperature of 37° C.) of the same composition but containing 125 mM KCl and no NaCl.

After a series of rinsings, rest periods and readjustment of the tension, the maximum contraction of each segment is determined by a new depolarisation with the KPSS solution.

After a new rest period during which the tension at 1 g is constantly readjusted, the following compounds are added into the isolated organ bath mepyramine (1 μM), atropine (1 μM), indometacine (3 μM), LNA (30 μM), captopril (10 μM), DL-thiorphan (1 μM) and nifedipine (0.1 μM). 20 minutes afterwards, the molecule to be tested or the solvent of the molecule is added into the isolated organ bath. The molecules are studied at 10 μM; if a molecule presents a sufficient degree of activity, it is studied at lower concentrations (e.g.: 0.1-0.01 μM).

After 15 minutes' incubation, the vein segments are contracted by the addition of increasing concentrations of des-Arg¹⁰-Kallidin (0.1 nM to 30,000 nM) in the container.

The EC₅₀ values (effective concentrations of agonist required to produce 50% of the maximum response obtained with the KPSS) are calculated by the least squares method.

The pK_{B}=[−log K/_{B}] is obtained from the equation

K_{B}=[A]/(concentration ratio−1)

wherein [A] is the concentration of antagonist and the (concentration ratio) represents the ratio between the EC₅₀ in the presence of antagonist, and the EC₅₀ in the absence of antagonist.

In accordance with this test, the compounds according to the invention cited in the description have a pK_{B} of between 7 and 9.

The compounds of the present invention are useful for the treatment of various forms of pain, such as inflammatory hyperalgesia, allodynia, neuropathic pain combined for example with diabetes, with neuropathies (constriction of the sciatic nerve, lumbar pains), with any form of traumatism, with a surgical operation (tooth extraction, removal of the tonsils), with an interstitial cystitis, with an inflammatory illness of the colon, or with a cancer.

Furthermore, it was verified that some of the compounds of the present invention significantly reduce the migration of neutrophils induced by an intrapleural injection of carrageen in the mouse according to the methods described previously (A. L. F. Sampaio, G. A. Rae, & M. G. M. O. Henriques, Participation of endogenous endothelins in delayed eosinophil and neutrophil recruitment in mouse pleurisy. Inflamm. Res., 49. 170-176, 2000).

Thus, the compounds of the present invention can also be used for treating any pathology associated with a recruitment of neutrophils such as, for example, acute respiratory distress syndrome, psoriasis, chronic pulmonary obstructions, inflammatory illnesses of the colon, rheumatoid polyarthritis.

The activity of the compounds according to the invention, demonstrated during the biological tests, is indicative of analgesic properties and enables considering their use in therapeutics.

According to the invention, it is recommended to use the compounds defined by formula I, as well as their salts with non-toxic acids, as active principles of medicaments which are intended for a treatment in a mammal, notably in man, with regard to pain or certain illnesses which are generally characterised by a massive migration of neutrophils.

The following can be cited amongst the illnesses which can be treated by means of an administration of a therapeutically effective amount of at least one of the compounds of formula I: inflammatory hyperalgesiae, neuropathic pain, pain associated with a traumatism or with a cancer, inflammatory illnesses of the colon, rheumatoid polyarthritis, psoriasis, chronic pulmonary obstructions, or acute respiratory distress syndrome.

The dosage of the active principle depends upon the mode of administration and upon the type of pathology; it is generally between 0.05 and 10 mg/kg. As a function of the treatment sought after, it will be possible for the compounds of formula I or their salts to be combined with other active principles, and will be formulated with commonly used excipients. With the aim of obtaining a rapid action, notably when treating pain, the mode of administration of the medicament will preferably be done by injection, e.g. via the intramuscular or subcutaneous route.

1. An N-(phenylsulphonyl) glycine compound, characterised in that it is selected from the whole which is made up of
i) compounds of formula: 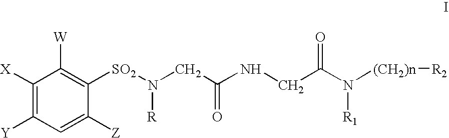
in which
W represents a chlorine atom,
X represents a hydrogen atom, a methyl group or a chlorine atom,
Y and Z independently each represent a hydrogen atom or a chlorine atom, or
X and W or X and Y, together with the carbon atoms to which they are bound, form a phenyl ring,
R represents a hydrogen atom, an allyl group or a C₁-C₄ alkyl group which is non-substituted or substituted with a phenyl group, a methoxy group, a pyridinyl group, a carboxamide group or an N-methylcarboxamide group,
R₁ represents a hydrogen atom, a C₁-C₄ alkyl group or
a (CH₂)ₘ—R′₂ group
n and m independently each represent 1, 2, 3 or 4,
R₂ and R′₂ independently each represent 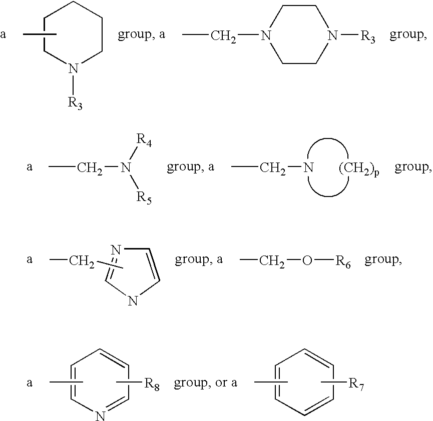
group, and
R₃ represents a hydrogen atom or a C₁-C₄ alkyl group,
R₄ represents a hydrogen atom, a COCH₃ group, a COOCH₃ group, or a C₁-C₄ alkyl group,
R₅ represents a hydrogen atom or a C₁-C₄ alkyl group, which is non-substituted or substituted with a phenyl group,
R₆ represents a hydrogen atom or a CONHC₂H₅ group,
R₇ represents a hydrogen atom, 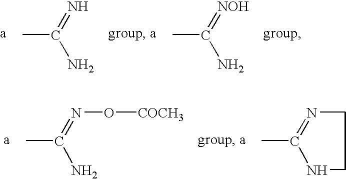
group or a CONHCH₃ group,
R₈ represents a hydrogen atom, an NH₂ group, or a C₁-C₄ alkyl group, and
p=4, 5 or 6,
i) addition salts of the compounds of formula I above with an acid.

2. The compound according to claim 1, characterised in that R represents a phenylmethyl group.

3. The compound according to claim 1, characterised in that R represents a C₁-C₄ alkyl group.

4. The compound according to one of claims 1 to 3, characterised in that:
R₁ represents a hydrogen atom, a C₁-C₄ alkyl group or
a (CH₂)ₘ—R′₂ group,
m represents 1, 2, 3 or 4,
R′₂ represents 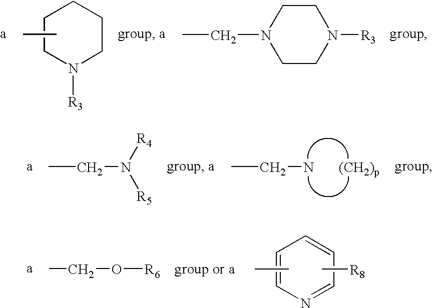
group, and
R₃ represents a hydrogen atom or a C₁-C₄ alkyl group,
R₄ represents a hydrogen atom, a COCH₃ group, a COOCH₃ group, or a C₁-C₄ alkyl group,
R₅ represents a hydrogen atom or a C₁-C₄ alkyl group, which is optionally substituted with a phenyl group,
R₆ represents a hydrogen atom or a CONHC₂H₅ group,
R₈ represents a hydrogen atom, and
p=4, 5 or 6.

5. The compound according to one of claims 1 to 4, characterised in that:
R₂ represents 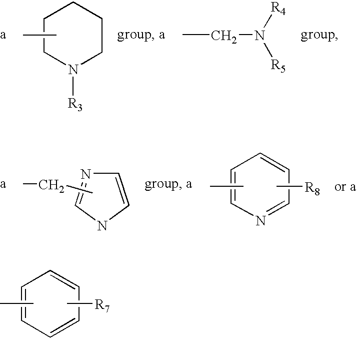
group, and
R₃ represents a hydrogen atom,
R₄ represents a hydrogen atom or a C₁-C₄ alkyl group, preferably a methyl group,
R₅ represents a hydrogen atom or a C₁-C₄ alkyl group, preferably a methyl group,
R₇ represents a hydrogen atom, 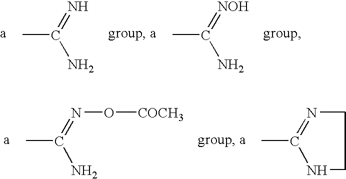
or a CONHCH₃ group, and
R₈ represents a hydrogen atom or an NH₂ group.

6. The compound according to one of claims 1 to 5, characterised in that R₁ or R₂ comprise an “amidinyl” group in their structure.

7. The compound according to claim 6, characterised in that R₂ represents a phenylamidine group.

8. The compound according to claim 6, characterised in that R₂ comprises a 2-imidazolyl group.

9. The compound according to one of claims 1 to 5, characterised in that it is selected from the whole which is made up of the following compounds:
N-[2-[[[4-(aminoiminomethyl)phenyl]methyl][4-(1-pyrrolidinyl)butyl]amino]2-oxoethyl]-2-[[(2,4-dichloro-3-methylphenyl)sulphonyl](2-phenylethyl)amino]acetamide, dihydrochloride,
N-[2-[[[4-(aminoiminomethyl)phenyl]methyl][3-(dimethylamino)propyl]amino]-2-oxoethyl]-2-[[(2,4-dichloro-3-methylphenyl)sulphonyl](2-phenylethyl)amino]acetamide, dihydrochloride,
N-[2-[[[4-(aminoiminomethyl)phenyl]methyl](4-piperidinylethyl)amino]-2-oxoethyl]-2-[[(2,4-dichloro-3-methylphenyl)sulphonyl](2-phenylethyl)amino]acetamide, bis trifluoroacetate,
N-[2-[(4-aminobutyl)(4-piperidinylmethyl)amino]-2-oxoethyl]-2-[[(2,4-dichloro-3-methylphenyl)sulphonyl](2-phenylethyl)amino]acetamide, bis trifluoroacetate,
2-[[(2,4-dichloro-3-methylphenyl)sulphonyl](2-phenylethyl)amino]-N-[2-[[[4-(4,5-dihydro-1H-imidazol-2-yl)phenyl]methyl][3-(dimethylamino)propyl]amino]-2-oxoethyl]acetamide, dihydrochloride,
2-[[(2,4-dichloro-3-methylphenyl)sulphonyl]methylamino]-N-[2-[[[4-(4,5-dihydro-1H-imidazol-2-yl)phenyl]methyl]methylamino]-2-oxoethyl]acetamide, hydrochloride, and
2-[[(2,4-dichloro-3-methylphenyl)sulphonyl](2-phenylethyl)amino]-N-[2-[[3-(1H-imidazol-5-yl)propyl][4-(1-pyrrolidinyl)butyl]amino]-2-oxoethyl]acetamide, dihydrochloride.

10. A pharmaceutical composition, characterised in that it contains, in combination with at least one physiologically acceptable excipient, at least one compound of formula I according to one of claims 1 to 9, or one of its addition salts with an acid.

11. Use of a compound of formula I or of one of its addition salts with an acid, for preparing a medicament intended for treating pain.

12. Use of a compound of formula I or of one of its addition salts with an acid, for preparing a medicament intended for treating inflammatory illnesses.
